(19) United States
(12) Patent Application Publication
(10) Pub. No.: US 2026/0063626 A1
(43) Pub. Date: Mar. 5, 2026

(54) COMPARTMENTAL ANALYSIS OF EXTRACORPOREAL LIVER PERFUSION SYSTEMS

(71) Applicant: HepQuant, LLC, Denver, CO (US)

(72) Inventors: Gregory Thomas EVERSON, Englewood, CO (US); Michael P. MCRAE, Houston, TX (US)

(21) Appl. No.: 19/318,207

(22) Filed: Sep. 3, 2025

Related U.S. Application Data
(60) Provisional application No. 63/690,064, filed on Sep. 3, 2024.

(51) Int. Cl. G01N 33/53; A61M 60/113; A61M 60/30; A61M 60/523; G01N 33/58

(52) U.S. Cl. CPC G01N 33/5308; A61M 60/113; A61M 60/30; A61M 60/523; G01N 33/58

(57) ABSTRACT

Methods are provided for effectively distinguishing the abilities of a human liver and an extracorporeal liver in an extracorporeal liver perfusion system to extract cholate and, thus, measure relative liver function.

### CROSS-REFERENCES TO RELATED APPLICATIONS

This application claims the benefit of and priority to U.S. Provisional Application No. 63/690,064, filed Sep. 3, 2024, the entire contents of which are incorporated by reference herein.

### BACKGROUND

Annually in the US, over 300,000 patients are admitted with various forms of liver failure, requiring treatment in an acute setting. Patients in liver failure face a high risk of mortality. The efficacy of existing liver support options is limited.

Extracorporeal liver perfusion systems (ECS) are used to keep livers viable for transplant. Normothermic machine perfusion of deceased donor livers using ECS was found to decrease post-transplant early allograft dysfunction (EAD) and ischemic biliary complications (IBC) compared to ischemic cold storage of livers. Markmann et al., Impact of portable normothermic blood-based machine perfusion on outcomes of liver transplant, JAMA Surgery, 2022; 157(3): 189-198.

Recently, an extracorporeal liver perfusion system (ECS) was employed in a successful completion of an extracorporeal perfusion of a human brain-dead research donor (BDRD) using an extracorporeal (EC) liver: a genetically engineered porcine liver. The perfusion was performed using the EC liver and the ECS to enable circulation of the donor's blood through the porcine liver. Stable blood flow, pressure, and pH were maintained throughout the procedure along with good bile production. The perfusion was electively stopped at 72 hours per protocol. The 72 hour proof-of concept procedure was the longest perfusion to date using genetically engineered porcine organ designed for liver support. Press Release-eGenesis and OrganOx Announce Successful Use of a Genetically Engineered Porcine Liver with a Human Donor-Jan. 18, 2024. For some patients, utilizing an extracorporeal liver in an ECS system may provide time for recovery of the patient's liver or time to obtain a liver transplant.

The cholate SHUNT tests of liver function and physiology have been shown to be useful for monitoring liver function, as well as treatment effects, and predicting risk for clinical outcome in patients. The cholate SHUNT test quantifies hepatic functional impairment from the simultaneous clearance of cholate from the systemic and portal circulations for the purpose of monitoring treatment effects or for predicting risk for clinical outcome. Test parameters, derived from a noncompartmental minimal model (MM), are reproducible and reliable (McRae et al., 2021, Translational Research, Volume 252, Pages 53-63).

U.S. Pat. No. 8,613,904, Everson et al., discloses cholate SHUNT test methods for evaluating liver function in a patient comprising obtaining patient serum samples following administration of two distinguishable stable isotope labeled cholate compounds, laborious sample processing and analysis of patient serum samples utilizing GC-MS.

U.S. Pat. No. 8,778,299, Everson, discloses cholate SHUNT test methods for evaluating liver function comprising obtaining patient serum samples following administration of two distinguishable stable isotope labeled cholate compounds, processing and analysis of patient serum samples utilizing HPLC-MS. Methods for determining portal hepatic filtration rate (portal HFR, FLOW) from oral administration of a distinguishable agent are also provided.

U.S. Pat. No. 9,091,701, Everson, discloses methods for determining liver function and obtaining a Disease Severity Index (DSI) value in a patient comprising obtaining patient serum samples following administration of two distinguishable stable isotope labeled cholate compounds, processing and analysis of patient serum samples utilizing HPLC-MS.

U.S. Pat. No. 8,961,925, Everson, discloses methods for performing the STAT test comprising oral administration of a distinguishable agent and measuring the distinguishable compound in a single blood or serum sample following using HLPC-MS. Methods of estimating portal hepatic filtration rate (portal HFR) from STAT values are also provided.

US Pat. Appl. Pub. No. US 2021/0318274 A1, Everson et al., discloses improved methods for blood or serum sample preparation, analyte detection and quantification which may be applied to one or more of the SHUNT, FLOW, STAT, and DSI liver function tests.

US Pat. Appl. Pub. No. US 2024/0175881, McRae et al., discloses simplified methods for measuring liver function including cholate liver function tests SHUNT V1.0, SHUNT V1.1, SHUNT V2.0, and DuO analysis methods.

Methods for determining relative liver function in a patient liver and an extracorporeal liver in an extracorporeal liver perfusion system (ECS) system are desirable.

### SUMMARY

Methods are provided for effectively distinguishing the abilities of a human liver and an extracorporeal (EC) liver in an extracorporeal liver perfusion system (ECS) to extract cholate and, thus, measure relative liver function.

A method is provided for determining hepatic clearance in a system comprising (i) a human patient comprising a native liver supported on (ii) an extracorporeal liver perfusion system (ECS) comprising an (iii) extracorporeal liver, the method comprising
determining compartment volumes (L) of the patient systemic circulation(S), the extracorporeal liver perfusion system (E), the extracorporeal liver (X), and the human patient native liver (H);
deriving or measuring blood flow rates q (L/min) between the compartments;
obtaining blood or serum samples collected at one or more or two or more time points within 180 minutes of intravenous administration of a dose of a first distinguishable cholate compound (D_{IV}) to the systemic circulation of the patient(S), and optional simultaneous oral or nasogastric (NG) tube administration of a dose of a second distinguishable cholate compound (Dₒᵣₐₗ) to the patient;
wherein the blood or serum samples were simultaneously collected from each of the patient systemic circulation (B_{S}); the extracorporeal liver perfusion system (B_{E}); and inferior vena cava (IVC) outflow of the EC liver (B_{X});
measuring the first distinguishable cholate concentration and optionally the second distinguishable cholate concentration in each of the blood or serum samples; and
calculating the simultaneous systemic clearance of the intravenously administered first distinguishable cholate of the patient liver (Cl_{H}) and of the extracorporeal liver (Clx) in the extracorporeal liver perfusion system (ECS).

In some cases, the method further comprises calculating portal clearance of the orally or nasogastric administered second distinguishable cholate of the patient liver (Cl_{H}) and of the extracorporeal liver (Clx) in the extracorporeal liver perfusion system (ECS).

In some cases, the blood or serum samples were collected at one or more or two or more time points within about 120 min, about 90 min, about 60 min, about 45 min, or about 20 minutes after the intravenous administration of the first distinguishable cholate compound to the systemic circulation of the patient(S).

In some cases, the deriving or measuring flow rates (L/min) between each of the compartments patient systemic circulation (S), patient human liver (H), ECS (E), and extracorporeal liver (X) comprises a system of first-order ordinary differential equations 1 to 4, respectively:

$$\frac{{dC}_{S}}{dt} = \frac{1}{{V}_{S}} ( - [ {q}_{SH} + {q}_{SE} ] {C}_{S} + {q}_{HS} \cdot {C}_{H} + {q}_{ES} \cdot {C}_{E} ) , \qquad Eqn . 1$$

$$\frac{{dC}_{H}}{dt} = \frac{1}{{V}_{H}} ( - [ {Cl}_{H} + {q}_{HS} ] {C}_{H} + {q}_{SH} \cdot {C}_{S} ) , \qquad Eqn . 2$$

$$\frac{{dC}_{E}}{dt} = \frac{1}{{V}_{E}} ( - [ {q}_{ES} + {q}_{EX} ] {C}_{E} + {q}_{XE} \cdot {C}_{X} + {q}_{SE} \cdot {C}_{S} ) , and \qquad Eqn . 3$$

$$\frac{{dC}_{X}}{dt} = \frac{1}{{V}_{X}} ( - [ {Cl}_{X} + {q}_{XE} ] {C}_{X} + {q}_{EX} \cdot {C}_{E} ) , \qquad Eqn . 4$$

wherein
V is the volume (L) in each of the patient systemic circulation (V_{S}), patient human liver (V_{H}), ECS (VE), and extracorporeal liver (V_{X}) compartments; C is the blood concentration (mM) of first distinguishable cholate in each of the patient systemic circulation (C_{S}), patient human liver (C_{H}), ECS (C_{E}), and extracorporeal liver (C_{X}) compartments; q is the blood flow rate between compartments (L/min); Cl_{H} is the hepatic clearance (L/min) of the human liver, and Cl_{X} is the hepatic clearance (L/min) of the extracorporeal liver.

In some cases, q_{SH} is patient systemic inflow to human liver (hepatic arterial inflow+portal venous inflow), q_{HS} is outflow from human liver back to systemic circulation, q_{SE} is inflow from patient systemic circulation to ECS, q_{ES} is outflow from ECS to patient systemic circulation, q_{XE} is inferior vena cava (IVC) blood outflow from EC liver back to ECS, q_{EX,PV} is portal venous (PV) blood inflow to EC liver from ECS, q_{EX,HA} is hepatic arterial inflow to EC liver from ECS, and q_{EX} is total blood inflow to EC liver from ECS.

A method of determining hepatic clearance in a system is provided, the system comprising (i) a human patient without a native liver supported on (ii) an extracorporeal liver perfusion system (ECS) comprising an (iii) extracorporeal liver, the method comprising
determining compartment volumes (L) of the patient systemic circulation(S), the extracorporeal liver perfusion system (E), and the extracorporeal liver (X); deriving or measuring blood flow rates q (L/min) between the compartments;
obtaining blood or serum samples collected at one or more or two or more time points within 180 minutes of intravenous administration of a dose of a first distinguishable cholate compound (D_{IV}) to the systemic circulation of the patient(S), and optional simultaneous oral or nasogastric (NG) tube administration of a dose of a second distinguishable cholate compound (Dₒᵣₐₗ) to the patient, wherein the blood or serum samples were simultaneously collected from each of the patient systemic circulation (B_{S}); the extracorporeal liver perfusion system (B_{E}); and inferior vena cava (IVC) outflow of the EC liver (B_{X});
measuring the first distinguishable cholate concentration and optionally the second distinguishable cholate concentration in each of the blood or serum samples; and
calculating the systemic clearance of the intravenously administered first distinguishable cholate of the extracorporeal liver (Clx) in the extracorporeal liver perfusion system (ECS).

In some cases, the method further comprises calculating simultaneous portal clearance of the orally or nasogastric administered second distinguishable cholate of the extracorporeal liver (Clx) in the extracorporeal liver perfusion system (ECS).

In some cases, the blood or serum samples were collected at one or more or two or more time points within about 120 min, about 90 min, about 60 min, about 45 min, or about 20 minutes of administering the first distinguishable cholate compound to the systemic circulation of the patient (S) via intravenous administration.

In some cases, the deriving or measuring flow rates (L/min) between each of the compartments patient systemic circulation (S), ECS (E), and extracorporeal liver (X) comprises a system of first-order ordinary differential equations 5 to 7, respectively:

$$\frac{{dC}_{S}}{dt} = \frac{1}{{V}_{S}} ( - {q}_{SE} {C}_{S} + {q}_{ES} \cdot {C}_{E} ) , \qquad Eqn . 5$$

$$\frac{{dC}_{E}}{dt} = \frac{1}{{V}_{E}} ( - [ {q}_{ES} + {q}_{EX} ] {C}_{E} + {q}_{XE} \cdot {C}_{X} + {q}_{SE} \cdot {C}_{S} ) , and \qquad Eqn . 6$$

$$\frac{{dC}_{X}}{dt} = \frac{1}{{V}_{X}} ( - [ {Cl}_{X} + {q}_{XE} ] {C}_{X} + {q}_{EX} \cdot {C}_{E} ) , \qquad Eqn . 7$$

wherein
V is the volume (L) in each of the patient or BDRD systemic circulation (V_{S}), the ECS (V_{E}), and the extracorporeal liver (V_{X}) compartments; C is the blood concentration (mM) of first distinguishable cholate in each of the patient systemic circulation (C_{S}), ECS (C_{E}), and extracorporeal liver (C_{X}) compartments; q is the blood flow rate between compartments (L/min); and Cl_{X} is the hepatic clearance (L/min) of the extracorporeal liver. In some cases, the q_{SE} is inflow from patient systemic circulation to ECS, q_{ES} is outflow from ECS to patient systemic circulation, q_{XE} is inferior vena cava (IVC) blood outflow from EC liver back to ECS, and q_{EX} is total blood inflow to EC liver from ECS.

A method of determining total systemic clearance (Cl_{Tot}) in a system is provided, the system comprising an extracorporeal liver perfusion system (ECS), a human patient having a native liver, and an extracorporeal (EC) liver, the method comprising:
receiving blood or serum samples collected from one or more of the ECS, the patient systemic circulation, and inferior vena cava (IVC) outflow of the EC liver at one or more, or two or more, time points within 180 minutes of intravenous administration to the patient of a first distinguishable cholate compound, and optional simultaneous oral or nasogastric administration to the patient of a second distinguishable cholate compound;
measuring distinguishable cholate concentrations (C) of the first distinguishable cholate compound and optionally the second distinguishable cholate compound in each of the blood or serum samples;
optionally measuring blood flow rates (q, L/min) in one or more of the ECS (E), the human patient systemic circulation(S), and the extracorporeal liver (X) in at least one of the time points; and
calculating total systemic clearance (Cl_{Tot}) as the intravenous clearance of first distinguishable cholate compound of the system.

In some cases, the calculating intravenous clearance of the first distinguishable cholate from the system comprises a method selected from the group consisting of SHUNT V1.0, SHUNT V1.1, and SHUNT V2.0 methods as disclosed herein.

In some cases, the method further comprises calculating systemic clearance in the ECS (Cl_{E}) comprising
measuring the ECS blood flow rate (q, L/min) as blood inflow rate from patient systemic circulation to the ECS (q_{SE}, L/min); and
calculating the first distinguishable cholate compound clearance in the ECS (Cl_{E}) as the q_{SE} times extraction efficiency of the ECS (E_{E}) by Eqn 10:

$${Cl}_{E} = {q}_{SE} \cdot {E}_{E} . \qquad Eqn . 10$$

In some cases, the extraction efficiency of the ECS (E_{E}) at one or more of the time points (t) is calculated based on the concentration of the distinguishable cholate compound in the ECS and flow rates in and out of the ECS.

In some cases, the extraction efficiency of the ECS (E_{E}) is calculated at the one or more of the time points (t) comprising Eqn. 8:

$${E}_{E} ( t ) = \frac{{C}_{S} ( t ) \cdot {q}_{SE} ( t ) - {C}_{E} ( t ) \cdot {q}_{ES} ( t )}{{C}_{S} ( t ) \cdot {q}_{SE} ( t )} , \qquad Eqn . 8$$

wherein
C_{E}(t) is the concentration of the first distinguishable cholate compound at time (t) in the ECS;
q_{ES}(t) is the outflow rate at time (t) from the ECS to the patient systemic circulation;
C_{S}(t) is the concentration of first distinguishable cholate compound in the patient systemic circulation at time (t); and
q_{SE}(t) is the inflow rate from the patient systemic circulation to the ECS at time (t).

In some cases, the method further comprises calculating systemic clearance of the extracorporeal (EC) liver comprising
measuring the EC liver blood flow rate (q, L/min) as the IVC blood outflow rate from the extracorporeal (EC) liver back to ECS (q_{XE}); and
calculating the systemic clearance of the extracorporeal liver (Cl_{X}) as q_{XE} times the extraction efficiency of the extracorporeal liver (E_{X}) by Eqn. 11:

$${Cl}_{X} = {q}_{XE} \cdot {E}_{X} . \qquad Eqn . 11$$

In some cases, the extraction efficiency of the EC liver (E_{X}) is calculated at one or more of the time points (t) comprising Eqn. 9:

$${E}_{X} ( t ) = \frac{{C}_{E} ( t ) \cdot {q}_{EX} ( t ) - {C}_{X} ( t ) \cdot {q}_{XE} ( t )}{{C}_{E} ( t ) \cdot {q}_{EX} ( t )} , \qquad Eqn . 9$$

wherein
C_{E}(t) is the concentration of the first distinguishable cholate compound at time (t) in the ECS;
q_{EX}(t) is the total blood inflow to EC liver from the ECS at time (t);
C_{X}(t) is the concentration of the first distinguishable cholate compound at time (t) in the IVC outflow of the EC liver; and
q_{XE}(t) is IVC blood outflow from EC liver back to ECS. In some cases, the one or more time points is within about 180 min, 120 min, 90 min, 60 min, 30 min, or 20 min of the intravenous administration.

In some cases, the method further comprises
calculating the systemic clearance of the human liver (Cl_{H}) as the total clearance (Cl_{Tot}) minus the clearance of the ECS (Cl_{E}) comprising Eqn. 12:

$${Cl}_{H} = {Cl}_{Tot} - {Cl}_{E} . \qquad Eqn . 12$$

In some cases, the method further comprises in one of more of the ECS system, the patient, and the extracorporeal liver estimating an area under the curve of the blood or serum concentrations of the intravenously administered distinguishable cholate compound (AUCiv); and optionally estimating an area under the curve of the blood or serum concentrations of the orally or NG administered distinguishable cholate compound (AUCoral).

In some cases, the method further comprises estimating the AUCiv in one of more of the ECS system, the patient, and the extracorporeal liver comprises exponential fitting the intravenous concentration data to a systemic cholate clearance curve comprising fast, moderate, and slow phases of clearance over about 180 min or more after the administration of the intravenous dose.

In some cases, the method further comprises calculating systemic hepatic filtration rate (HFR_{S}) in one of more of the ECS system, the patient, and the extracorporeal liver from the measured systemic clearance adjusted for body weight (units mL min⁻¹ kg⁻¹) by Equation 26,

$${HFR}_{S} = \frac{{D}_{IV}}{{AUC}_{IV} \cdot BW} , \qquad Eqn . 26$$

where D_{IV} is the administered intravenous dose of the first distinguishable cholate, and BW is the patient body weight.

In some cases, the method further comprises calculating portal hepatic filtration rate (HFRₚ) in one of more of the ECS system, the patient, and the extracorporeal liver from the measured systemic clearance body weight (units mL min⁻¹ kg⁻¹) by Equation 25:

$${HFR}_{P} = \frac{{D}_{PO}}{{AUC}_{Oral} \cdot BW} , \qquad Eqn . 25$$

where D_{PO} is the administered oral or nasogastric cholate dose of second distinguishable cholate, and BW is the patient body weight.

In some cases, the first and optional second distinguishable cholate compounds are independently distinguishable cholic acids, cholic acid conjugates, or cholic acid analogs, wherein the first and optional second distinguishable cholate compounds are different.

In some cases, the first and optional second distinguishable cholic acids are independently isotopically labeled cholic acids.

In some cases, the isotopically labeled cholic acids are stable isotope labeled cholic acid.

In some cases, the first and second isotopically labeled cholic acids are different stable isotope labeled cholic acid.

In some cases, the stable isotope labeled cholic acids are selected from the group consisting of cholic acid-2,2,4,4-D4 (D4-CA; CA-D4), 24-¹³C-cholic acid (¹³C-CA), 2,2,3,4,4-d₅ cholic acid (D₅-CA), and 3,6,6,7,8,11,11,12-d8 cholic acid (D8-CA).

### BRIEF DESCRIPTION OF THE DRAWINGS

FIG. 1 shows a schematic of a compartmental model of the simultaneous co-clearance of intravenously administered distinguishable cholate compound by a human patient liver and an extracorporeal liver in an extracorporeal liver perfusion system (ECS).

FIG. 2 shows a schematic of a compartmental model of the clearance of intravenously administered distinguishable cholate compound by an extracorporeal liver in an extracorporeal liver perfusion system (ECS) after having removed the human patient's liver.

FIG. 3A shows graphs of compartmental model results for days 1, 2, and 3 on the ECS. The lines represent the model-predicted concentrations of 13C-CA over 60 minutes. Blue line=systemic compartment (BDRD), red line=extracorporeal liver perfusion system (ECS) compartment, orange line=pig liver compartment (PIG).

FIG. 3B shows a prior art example intravenous clearance graph of distinguishable cholate concentration vs. time calculated by cholate SHUNT V2.0 liver function test exponential fits to systemic clearance curve of an intravenously administered distinguishable cholate (13C-CA) compared to Minimal Model (MM) systemic clearance curve (5 data points). A noncompartmental analysis method involving the exponential fits of systemic cholate clearance using only a single IV (e.g., 20 min. 13C-CA concentration) timepoint. In SHUNT V2.0 cholate liver function test, a second sample at 60 min can also be analyzed. The exponential fits split the curve into three sections or phases: fast, moderate, and slow (0 to 20 min., 20-45 min., and 45-180 min. for Y₀, Y₁, and Y₂, respectively).

FIG. 3C shows a prior art example of cholate SHUNT V2.0 liver function test which calculates oral (PO) and intravenous (IV) distinguishable cholate clearances using samples collected at two time points: e.g., 20 and 60 minutes.

FIG. 4 shows a prior art schematic of a compartmental model of dual cholate clearance in a patient having a native liver describing the transfer of cholate between systemic, portal, and liver compartments in the patient. D_{IV}=intravenous dose of 13C cholate (SHUNT V2.0); D_{PO}=oral dose of d4-CA (SHUNT V2.0 or DuO); q_{SP}=splanchnic arterial flow rate; q_{PS}=portal systemic shunt flow rate; q_{PL}=portal venous flow rate; q_{LS}=hepatic return to systemic circulation flow rate; q_{SL}=hepatic arterial flow rate; Cl_{H}=hepatic clearance. Reprinted from McRae, M. P. et al., 2023, Compartmental model describing the physiological basis for the HepQuant SHUNT test, Translational Research, Volume 252, Pages 53-63, with permission from Elsevier.

FIG. 5 shows a schematic of the components and flow sampling points in a representative prior art extracorporeal liver perfusion system (ECS) supporting a patient and comprising an extracorporeal liver.

FIG. 6 shows a graph of 13C-cholate clearance following administration to subjects as concentration [13C-CA] (micromoles) in blood samples vs time (min) in healthy controls (n=50) (solid line), a brain dead research donor (BDRD) before ECS (a), and on day 1 (b), day 2 (c), and day 3 (d) at 20 and 60 min after 13C-CA administration in the extracorporeal liver perfusion system (ECS) supporting the BDRD and comprising an extracorporeal liver.

FIG. 7 shows a graph of Systemic Hepatic Filtration Rates (HFRs) calculated in a non-compartmental model in an ECS with an extracorporeal (EC) liver (pig liver) supporting a brain-dead research donor (BDRD3) from day 0 to day 4 for each of (A) Cl 13C-CA, total, (B) Cl 13C-CA, BDRD, and (C) Cl 13C-CA, Pig Liver calculated using a noncompartmental analysis of the ECS in a brain-dead research donor (BDRD) with pig liver. The solid bar represents Systemic HFR range in healthy controls.

### DETAILED DESCRIPTION OF THE INVENTION

### Definitions

The singular forms “a”, “an” and “the” are intended to include the plural forms as well, unless the context clearly indicates otherwise.

The term “and/or” refers to and encompasses any and all possible combinations of one or more of the associated listed items.

The term “about,” when referring to a measurable value such as an amount of a compound, dose, time, temperature, and the like, is meant to encompass variations of +/−10% of the specified amount. For example, the phrase “about 60 minutes” refers to 60 minutes+/−6 minutes. In some cases, the term “about” refers to 5%, 1%, 0.5%, or even 0.1% of the specified amount.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs. In the event of conflicting terminology, the present specification is controlling.

The term “accuracy” (measurement) when used herein refers to closeness of agreement between a measured quantity value and a true quantity value of a measurand.

The term “acceptability” as used herein is based on individual criteria that set minimal operational characteristics for a measurement procedure.

The term “precision” as used herein refers to closeness of agreement between independent test/measurement results obtained under stipulated conditions.

The term “trueness” as used herein refers to the closeness of agreement between the expectation of a test result or a measurement result and a true value.

The term “measurand” is used when referring to the quantity intended to be measured instead of analyte (component represented in the name of a measurable quantity).

The term “verification” as used herein focuses on whether specifications of a measurement procedure can be achieved, whereas the term “validation” verifies that the procedure is fit for an intended purpose.

The term “measurement procedure” refers to a detailed description of a measurement according to one or more measurement principles and to a given measurement method, based on a measurement model and including any calculation to obtain a measurement result.

As used herein “clearance” can mean the removing of a substance from one place to another.

As used herein, the term “simultaneously” when referring to 2 or more events refers to occurring within 10 minutes or less, 5 minutes, or within about 3 minutes of each other.

As used herein the terms, “patient”, “subject” or “subjects” include but are not limited to humans, the term may also encompass other mammals, or domestic or exotic animals, for example, dogs, cats, ferrets, rabbits, pigs, horses, cattle, birds, or reptiles.

The term “AUC” refers to the area under the individualized oral or intravenous clearance curves over time, e.g., over 90 minutes, 180 minutes, or infinity. The AUCiv refers to the area under the curve of blood or serum concentrations of an intravenously administered distinguishable cholate compound. The term AUCoral or AUCpo refers to the area under the curve of blood or serum concentrations of an orally administered distinguishable cholate compound.

The acronym “HALT-C” refers to the Hepatitis C Antiviral Long-term Treatment against Cirrhosis trial. The HALT-C trial was a large, prospective, randomized, controlled trial of long-term low dose peg interferon therapy in patients with advanced hepatitis C who had not had a sustained virologic response to a previous course of interferon-based therapy. An NIH-sponsored Hepatitis C Antiviral Long-Term Treatment against Cirrhosis (HALT-C) Trial examined whether long-term use of antiviral therapy (maintenance treatment) would slow the progression of liver disease. In noncirrhotic patients who exhibited significant fibrosis, effective maintenance therapy was expected to slow or stop histological progression to cirrhosis as assessed by serial liver biopsies. However, tracking disease progression with biopsy carries risk of complication, possibly death. In addition, sampling error and variation of pathologic interpretation of liver biopsy limits the accuracy of histologic assessment and endpoints. The histologic endpoint is less reliable because advanced fibrosis already exists and changes in fibrosis related to treatment or disease progression cannot be detected. Thus, standard endpoints for effective response to maintenance therapy in cirrhotic patients are prevention of clinical decompensation (ascites, variceal hemorrhage, and encephalopathy) and stabilization of liver function as measured clinically by Childs-Turcotte-Pugh (CTP) score. However, clinical endpoints and CTP score were known to be insensitive parameters of disease progression. Dual isotope techniques employing distinguishable cholates were used in development of the SHUNT test and used in conjunction with the HALT-C trial.

The term “SHUNT test” refers to a previously disclosed QLFT (quantitative liver function test) used as a comprehensive assessment of hepatic blood flow and liver function. The SHUNT test is used to determine clearance of orally and intravenously administered distinguishable cholates in subjects with and without chronic liver disease. SHUNT fraction or percent quantifies the spillover of the PO d4-cholate into the systemic circulation from the ratio of the clearance of the intravenously administered 13C-cholate to the clearance of the orally administered d4-cholate. In the SHUNT test, at least 5 blood samples are analyzed which have been drawn from a patient at intervals over a period of at least about 90 minutes after oral and intravenous administration of differentiable cholates. The SHUNT test is disclosed in Everson et al., U.S. Pat. No. 8,613,904, which is incorporated herein by reference. These studies demonstrated reduced clearance of cholate in patients who had either hepatocellular damage or portosystemic shunting. The “SHUNT test value” refers to a number (in %). The term “SHUNT %” represents a quantitative measurement of portal-systemic shunting. SHUNT % is a measurement of the percentage of spillover of the orally administered d4-cholate. The first-pass hepatic elimination of cholate in percent of orally administered cholate is defined as (100%-SHUNT). SHUNT test methods are disclosed in U.S. Pat. Nos. 8,613,904, 9,639,665, 8,778,299, 9,417,230, and 10,215,746, each of which is incorporated herein by reference in its entirety. Analysis of samples for stable isotopically labeled cholates is performed by, e.g., GC-MS, following sample derivitization, or LC-MS, without sample derivitization, or LC-MS/MS, or MS/MS. The ratio of the AUCs of orally to intravenously administered distinguishable cholate compound, corrected for administered doses, defines the cholate shunt. The cholate shunt can be calculated using the formula: AUCₒᵣₐₗ/AUCᵢᵥ×Doseᵢᵥ/Doseₒᵣₐₗ×100%, wherein AUCₒᵣₐₗ is the area under the curve of the serum concentrations of the orally administered distinguishable cholate compound and AUCiv is the area under the curve of the intravenously administered distinguishable cholate compound.

The SHUNT test allows measurement of first-pass hepatic elimination of bile acids from the portal circulation. Flow-dependent, first pass elimination of bile acids by the liver ranges from about 60% for unconjugated dihydroxy, bile acids to about 95% for glycine-conjugated cholate. Free cholate, used herein has a reported first-pass elimination of approximately 80% which agrees closely with previously observed first pass elimination in healthy controls of about 83%. After uptake by the liver, cholic acid is efficiently conjugated to either glycine or taurine and secreted into bile. Physicochemically cholic acid may be easily separated from other bile acids and bile acid or cholic acid conjugates, for example, by using chromatographic methods.

The term “Cholate Elimination Rate”, kₑₗᵢₘ min⁻¹ represents the first phase of elimination of the intravenously administered 13C-cholate, calculation from Ln/linear regression of [13C-cholate] versus time (using only the 5- and 20-minute time points). Intravenously administered 13C-cholate is rapidly delivered to the liver via the hepatic artery. In contrast, the same 13C-cholate slowly transits to the liver via the portal vein due to the capacitance of the splanchnic vascular bed. Thus, the first phase of cholate elimination is more dependent upon clearance from the hepatic artery than from portal vein.

The term “Volume of distribution”, V_{d}, (L kg⁻¹) represents the body's volume into which cholate is distributed.

The acronym “IV” or “iv” refers to intravenous route of administration.

The acronym “PO” refers to per oral route of administration.

The acronym “PHM” refers to perfused hepatic mass.

The acronym “SF” refers to shunt fraction, for example, as in liver SF, or cholate SF.

The acronym “ROC” refers to receiver operating characteristic. The ROC curve is a graphical plot which illustrates performance of a binary classifier system as its discrimination threshold is varied. It is created by plotting the fraction of true positives out of the positives (TPR=true positive rate) vs. the fraction of false positives out of the negatives (FPR-false positive rate), at various threshold settings. Sensitivity is the probability of a positive test result, or of a value above a threshold, among those with disease. Sensitivity is defined as the true positive rate (TPR): TPR=TP/P=TP/(TP+FN). False positive rate (FPR) is FPR=FP/N=FP/(FP+FN). Accuracy (ACC) is defined as ACC=(TP+TN)/(P+N). Specificity is the probability of a negative test result, or a value below a threshold, among those without disease. Specificity (SPC), or true negative rate (TN) is defined as SPC=TN/N=TN/(FP+TN)=1-FPR. Positive prediction value (PPV) is defined as: PPV=TP/(TP+FP). Negative predictive value (NPV) is defined as NPV=TN/(TN+FN).

The c-statistic is the area under the ROC curve, or “AUROC” (area under receiver operating characteristic curve) and ranges from 0.5 (no discrimination) to a theoretical maximum of 1 (perfect discrimination).

The terms “treating” or “treatment” of a disease state or condition includes: (i) preventing the disease state or condition, i.e., causing the clinical symptoms of the disease state or condition not to develop in a subject that may be exposed to or predisposed to the disease state or condition, but does not yet experience or display symptoms of the disease state or condition, (ii) inhibiting the disease state or condition, i.e., arresting the development of the disease state or condition or its clinical symptoms, or (iii) relieving the disease state or condition, i.e., causing temporary or permanent regression of the disease state or condition or its clinical symptoms.

The term “distinguishable cholate” or “distinguishable cholate compound” may be may any cholate compound that is distinguishable analytically from naturally occurring cholate in the blood or serum of a subject. The distinguishable cholate compound may be a labeled cholate compound or an unlabeled cholate compound. The distinguishable cholate compound may be a fluorescent moiety-labeled cholate compound. Various fluorescent probes are commercially available such as, e.g., fluorescein, Alexa Fluor dyes, quantum dots, and the like. The distinguishable cholate be an isotope labeled cholate compound. Distinguishable cholate compounds may be labeled with either stable isotopes (e.g., ¹³C, ²H, ¹⁸O) or radioactive isotopes (e.g., ¹⁴C, ³H). Distinguishable cholate compounds are commercially available and can be purchased (for example CDN Isotopes Inc., Quebec, CA).

The distinguishable cholate compound may be a stable isotope labeled cholate compound. In some cases, the distinguishable cholate may be selected from any known safe, non-radioactive stable isotope of cholic acid. In one specific aspect, the distinguishable cholate compound is 2,2,4,4-²H cholic acid, also known as cholic-acid-2,2,4,4-d₄ (D₄-CA). In another specific aspect, the distinguishable cholate compound is 24-¹³C cholic acid, also known as cholic acid-24-¹³C (¹³C-CA). In another specific aspect, the distinguishable compound is 2,2,3,4,4-²H cholic acid, also known as cholic acid-2,2,3,4,4-d₅ (D₅-CA).

In some embodiments, the distinguishable cholate compound may be selected from any of the following labeled compounds: cholic acid, any glycine conjugate of cholic acid, any taurine conjugate of cholic acid; chenodeoxycholic acid, any glycine conjugate of chenodeoxycholic acid, any taurine conjugate of chenodeoxycholic acid; deoxycholic acid, any glycine conjugate of deoxycholic acid, any taurine conjugate of deoxycholic acid; or lithocholic acid, or any glycine conjugate or taurine conjugate thereof. The distinguishable cholate compound may be selected from those described in WO 2021/207683 A1, HepQuant, LLC, Everson and Helmke, which is incorporated herein by reference in its entirety.

Cholates occur naturally and are not known to have any deleterious or adverse effects when given intravenously or orally in the doses used in the inventive or comparative tests herein. The serum cholate concentrations that are achieved by either the intravenous or oral doses are similar to the serum concentrations of bile acids that occur after the ingestion of a fatty meal. Because cholates are naturally occurring with a pool size in humans of 1 to 5 g, the typical 20 and 40 mg doses of labeled cholates used herein are unlikely to be harmful.

In some cases, the intravenous dose (Dose_{IV}) of first distinguishable cholate is from about 5 mg to about 100 mg, about 10 mg to about 50 mg, about 15 mg to about 30 mg, or about 20 mg. In some cases, the patient receives a 20 mg dose of cholic acid-24-¹³C (13C-CA) in an intravenous bolus. In some cases, the intravenous dose is diluted in and mixed with human albumin (e.g., 25% (w/v) human albumin in a buffered diluent such as sodium carbonate and optionally one or more stabilizers such as N-acetyl-D,L-tryptophan and caprylic acid). 25% human albumin is commercially available, for example as FLEXBUMIN®, Takeda Pharmaceuticals U.S.A., Inc. or ALBURX®, CSL Behring LLC. The human albumin may be isolated or recombinant human albumin. In some cases, a formulation of 20 mg Cholic Acid-24-¹³C in 5 cc 1 mEq/ml Sodium Bicarbonate is prepared and mixed with 5cc 25% human albumin prior to intravenous administration. In some cases, the intravenous dose of first distinguishable cholate is premixed with the human albumin.

In some cases, the optional oral or nasogastric dose (Doseₒᵣₐₗ) of second distinguishable cholate is from about 10 mg to about 200 mg, about 20 mg to about 100 mg, about 25 mg to about 55 mg, or about 40 mg. In some cases, the oral or nasogastric dose of second distinguishable cholate compound, e.g., 40 mg cholic-acid-2,2,4,4-d4 (4D-CA), is dissolved in aqueous NaHCO₃, and optionally mixed with diluent. The diluent may be water, sodium bicarbonate solution, non-citrus juice, and normal saline. In some cases, an oral solution including 2,2,4,4-2H-Cholic acid (40 mg) and Sodium Bicarbonate (600 mg) is dissolved in about 10 cc water prior to testing by mixing vigorously. The solution is stored in either the refrigerator or at room temperature. Just prior to administration, a diluent such as grape or apple (non-citrus) juice is added to the mixture and administered to the patient orally or by nasogastric tube.

The term “oral cholate clearance” (Clₒᵣₐₗ) refers to clearance from the body of a subject of an orally administered cholate compound as measured by a blood or serum sample from the subject. Oral cholate clearance is used as a measure of portal blood flow. Orally administered cholic acid is absorbed across the epithelial lining cells of the small intestine, bound to albumin in the portal blood, and transported to the liver via the portal vein. Approximately 80% of cholic acid is extracted from the portal blood in its first pass through the liver. Cholic acid that escapes hepatic extraction exits the liver via hepatic veins that drain into the vena cava back to the heart, and is delivered to the systemic circulation. The area under the curve (AUC) of peripheral venous concentration versus time after oral administration of cholic acid quantifies the fraction of cholic acid escaping hepatic extraction and defines “oral cholate clearance”.

The term “portal hepatic filtration rate”, “portal HFR”, “FLOW test” (HFRp) refers to oral cholate clearance (portal hepatic filtration rate; portal HFR) used as a measure of portal blood flow, or portal circulation, obtained from analysis of concentration of distinguishable cholate compound in, e.g., 5 blood samples, drawn from a subject over a period of, for example, about 90 minutes after oral administration of a distinguishable cholate compound, for example, a distinguishable cholate. The units of portal HFR value are typically expressed as mL/min/kg, where kg refers to kg body weight of the subject. “Portal HFR”, mL min⁻¹ kg⁻¹, may be used to model independent apparent hepatic clearance of an orally administered distinguishable cholate compound, such as, e.g., d4-cholate, adjusted for body weight, and calculated from dose/AUC. FLOW test methods are disclosed in U.S. Pat. Nos. 8,778,299, 9,417,230, and 10,215,746, each of which is incorporated herein by reference in its entirety.

Changes in portal circulation can be detected by cholate liver function tests, e.g., portal HFR, SHUNT % (e.g., SHUNT V1.0, SHUNT V1.1), first pass hepatic extraction of a distinguishable cholate, and DSI. A change in one or more of these parameters can detect a change in portal circulation. A cholate SHUNT test (SHUNT 1.0) with prior art minimal model data analysis was able to detect treatment effects in a clinical study. Lawitz, E., et al., BI 685509 improves hepatic function in subjects with Child-Pugh A cirrhosis and a liver stiffness measurement of >15 kPa: Results from the HepQuant SHUNT test. Hepatology, 2021(74): p. 1238A-1239A. The simpler cholate DuO and SHUNT V2.0 tests may be used to detect the same treatment effects.

The term “intravenous cholate clearance” (Clᵢᵥ) refers to clearance of an intravenously administered cholate compound. Intravenously administered cholic acid, bound to albumin, distributes systemically and is delivered to the liver via both portal venous and hepatic arterial blood flow. The AUC of peripheral venous concentration versus time after intravenous administration of cholic acid is equivalent to 100% systemic delivery of cholic acid. The ratio of the AUCs of orally to intravenously administered distinguishable cholic acids, corrected for administered doses, defines cholate shunt.

The term “Systemic HFR”, (HFRs), mL min⁻¹ kg⁻¹, refers to Systemic Hepatic Filtration Rate which can be used to model independent clearance of intravenously injected distinguishable cholate compound, e.g. 13C-cholate, adjusted for body weight, and calculated from dose/AUC.

The term “STAT test” (STAT) refers to an estimate of portal blood flow by analysis from one patient blood sample drawn at a defined period of time following oral administration of a differentiable cholate. In one aspect, the STAT test refers to analysis of a single blood sample drawn at a specific time point after oral administration of a differentiable cholate. The STAT test is simply the orally administered distinguishable cholate (e.g., d4-CA) concentration at 60 minutes normalized to 75 kg body weight by the calculation ([d4-CA]×(kg body weight/75 kg)). The value derived from the STAT test may be used by itself or in the estimation of portal HFR and DSI. In one specific aspect, the STAT test is a simplified convenient test intended for screening purposes that can reasonably estimate the portal blood flow (estimated flow rate) from a single blood sample taken at a single time point, such as, e.g., 60 minutes, after orally administered distinguishable cholate compound, such as, e.g., deuterated-cholate. In some embodiments, STAT, is the d4-cholate concentration in a blood sample obtained 60 minute after oral administration. STAT correlates well with DSI and can be used to estimate DSI. The STAT test value is typically expressed as a concentration, for example, micromolar (uM) concentration. STAT test methods are disclosed in U.S. Pat. Nos. 8,961,925, 10,222,366, each of which is incorporated herein by reference in its entirety. STAT test value may be used to estimate portal HFR, as provided in U.S. Pat. Nos. 8,961,925, and 10,222,366. A STAT test value in a patient may be used to estimate a DSI value in a patient, for example, as provided in US 2021/0318274 A1.

In some cases, estimated DSI from STAT can be obtained by the equation:

$$y = 9.4514 ln ( x ) + 21.12 ,$$

where x=STAT value (mM adjusted to 75 kg bodyweight), y=estimated DSI value, and R²=0.8499.

In some cases, the concentration of the distinguishable compound in the single specific sample (STAT value) can be converted into an estimated DSI value in the patient:

$$y = {A ( Ln x )}^{2} + B ( Ln x ) + C ,$$

y=estimated DSI value in the subject;
x=STAT value in the subject;
A=coefficient from 1 to 1.5;
B=coefficient from 9 to 10; and
C=constant from 19.5 to 22.

In some cases, the STAT value can be converted into an estimated portal HFR (FLOW) (mL/min/kg) value in the patient by the equation:

$$y = A ( x ) + C ,$$

wherein
x=LOG estimated portal HFR (FLOW) value (mL/min/kg) in the patient;
y=LOG STAT value (□M adjusted to 75 kg bodyweight) in the patient;
A=slope coefficient from 0.9 to 1.1; and
C=a constant from −0.05 to 0.05.

In some cases, the equation for converting the STAT value into an estimated portal HFR (FLOW) (mL/min/kg) value in the patient is y=0.9702x+0.0206.

In some cases, the equation for converting the STAT value into an estimated portal HFR (FLOW) (mL/min/kg) value in the patient is:

$$Ln ( x ) = 1.031 \times Ln ( y ) - 0.0212 ,$$

wherein;
X=estimated portal HFR value (mL/min/kg) in the patient; and
y=STAT value (□M adjusted to 75 kg bodyweight) in the patient.

The term “DSI test” (DSI) refers to Disease Severity Index test which is derived from one or more liver function test results based on hepatic blood flow. The DSI score is a function of the sum of cholate clearances from systemic and portal circulations adjusted to disease severity ranging from healthy subjects to end stage liver disease. DSI is a score without units representing a quantitative measurement of liver function. A disease severity index (DSI) value may be obtained in a patient by a method comprising (a) obtaining one or more liver function test values in a patient and/or and ECS, wherein the patient has or is at risk of liver failure and/or a chronic liver disease, wherein the one or more liver function test values are obtained from one or more liver function tests selected from the group consisting of SHUNT, portal hepatic filtration rate (portal HFR), and systemic hepatic filtration rate (systemic HFR); and (b) employing a disease severity index equation (DSI equation) to obtain a DSI value in the patient, wherein the DSI equation comprises one or more terms and a constant to obtain the DSI value, wherein at least one term of the DSI equation independently represents a liver function test value in the patient, or a mathematically transformed liver function test value in the patient from step; and the at least one term of the DSI equation is multiplied by a coefficient specific to the liver function test. DSI is an index, or score, that encompasses the cholate clearances from both systemic and portal circulations. DSI has a range from 0 (healthy) to 50 (severe end-stage disease) and can be calculated from oral and systemic HFRs. Based on the reproducibility of DSI values, the minimum detectable difference indicating a change in liver function in a subject may be about 1.5 points, about 2 points, or about 3 points. DSI test methods and equations are disclosed in U.S. Pat. Nos. 9,091,701, 9,759,731, 10,520,517, each of which is incorporated herein by reference in its entirety. A method of estimating a DSI value in a patient from a STAT test value is also provided herein.

The term “Hepatic Reserve” refers to percentage of maximum hepatic functional capacity measured by DSI, indexed hepatic reserve may be normalized to the DSI range in subjects of lean body mass. HR (algebraic) is simply an algebraic conversion of the DSI value in the subject: HR=[100−(2×DSI)]. Indexed HR is normalized against the results within a cohort of normal lean controls.

The term “RCA20” represents the amount of the intravenously administered distinguishable compound, for example, a distinguishable cholate compound such as 13C-CA, that remains in the circulation 20 minutes after the intravenous injection. RCA20 and Hepatic Reserve methods are disclosed in US2021/0318274, Everson et al., which is incorporated herein by reference in its entirety.

The term “Quantitative Liver Function Test” (QLFT), refers to assays that measure the liver's ability to metabolize or extract test compounds, can identify patients with impaired hepatic function at earlier stages of disease, and possibly define risk for cirrhosis, splenomegaly, and varices. One of these assays is the cholate shunt assay where the clearance of cholate is assessed by analyzing bodily fluid samples after exogenous cholate has been taken up by the body.

The term “Ishak Fibrosis Score” is used in reference to a scoring system that measures the degree of fibrosis (scarring) of the liver, which is caused by chronic necroinflammation. A score of 0 represents no fibrosis, and 6 is established fibrosis. Scores of 1 and 2 indicate mild degrees of portal fibrosis; stages 3 and 4 indicate moderate (bridging) fibrosis. A score of 5 indicates nodular formation and incomplete cirrhosis, and 6 is definite cirrhosis.

The term “Childs-Turcotte-Pugh (CTP) score” or “Child-Pugh score” refers to a classification system used to assess the prognosis of chronic liver disease as provided in Pugh et al., Transection of the oesophagus for bleeding oesophageal varices. Br J Surg 1973; 60:646-649, which is incorporated herein by reference. The CTP score includes five clinical measures of liver disease; each measure is scored 1-3, with 3 being the most severe derangement. The five scores are added to determine the CTP score. The five clinical measures include total bilirubin, serum albumin, prothrombin time international normalized ratio (PT INR), ascites, and hepatic encephalopathy. The CTP score is one scoring system used in stratifying the seriousness of end-stage liver disease. Chronic liver disease is classified into Child-Pugh class A to C, employing the added score. Child-Pugh class A refers to CTP score of 5-6. Child-Pugh class B refers to CTP score of 7-9. Child-Pugh class C refers to CTP score of 10-15. A website calculates post-operative mortality risk in patients with cirrhosis. http://mayoclinic.org/meld/mayomodel9.html

The term “Model for End-Stage Liver Disease (MELD) refers to a scoring system used to assess the severity of chronic liver disease. MELD was developed to predict death within three months of surgery in patients who had undergone a transjugular intrahepatic portosystemic shunt (TIPS) procedure patients for liver transplantation. MELD is also used to determine prognosis and prioritizing for receipt of a liver transplant. The MELD uses a patient's values for serum bilirubin, serum creatinine, and international normalized ratio for prothrombin time (INR) to predict survival. The scoring system is used by the United Network for Organ Sharing (UNOS) and Eurotransplant for prioritizing allocation of liver transplants instead of the older Child-Pugh score. See UNOS (2009-01-28) “MELD/PELD calculator documentation”, which is incorporated herein by reference. For example, in interpreting the MELD score in hospitalized patients, the 3 month mortality is: 71.3% mortality for a MELD score of 40 or more.

The term “standard sample” refers to a sample with a known concentration of an analyte used for comparative purposes when analyzing a sample containing an unknown concentration of analyte.

The term “chronic liver disease” refers to a chronic liver disease or condition. In some cases, the chronic liver disease or condition is selected from the group consisting of chronic hepatitis C (CHC), chronic hepatitis B, metabolic dysfunction-associated alcoholic liver disease (Met-ALD), alcoholic liver disease (ALD), steatotic liver disease (SLD), fatty liver disease, Alcoholic SteatoHepatitis (ASH), Alcoholic Hepatitis (AH), metabolic dysfunction-associated steatotic liver disease (MASLD), Non-Alcoholic Fatty Liver Disease (NAFLD), steatosis, metabolic dysfunction-associated steatohepatitis (MASH), Non-Alcoholic SteatoHepatitis (NASH), autoimmune liver disease, cryptogenic cirrhosis, hemochromatosis, Wilson's disease, alpha-1-antitrypsin deficiency, liver cancer, liver failure, cirrhosis, primary sclerosing cholangitis (PSC), and other cholestatic liver diseases.

The term “Chronic Hepatitis C” (CHC) refers to a chronic liver disease caused by viral infection and resulting in liver inflammation, damage to the liver and cirrhosis. Hepatitis C is an infection caused by a blood-borne virus that attacks the liver and leads to inflammation. Many people infected with hepatitis C virus (HCV) do not exhibit symptoms until liver damage appears, sometimes years later, during routine medical tests.

The term “Steatotic Liver Disease” (SLD) encompasses various etiologies of hepatic steatosis.

The term “Alcoholic SteatoHepatitis” (ASH) refers to a chronic condition of inflammation of the liver which is caused by excessive drinking. Progressive inflammatory liver injury is associated with long-term heavy intake of ethanol and may progress to cirrhosis.

The term “Metabolic dysfunction-Associated Steatohepatitis (MASH), formerly known as “Non-Alcoholic SteatoHepatitis” (NASH) refers to a serious chronic condition of liver inflammation, progressive from the less serious simple fatty liver condition called steatosis. Simple steatosis (alcoholic fatty liver) is an early and reversible consequence of excessive alcohol consumption. In people that don't drink much alcohol, the cause of fatty liver disease is less clear, but may be associated with factors such as obesity, high blood sugar, insulin resistance, or high levels of blood triglycerides. In certain cases, the fat accumulation can be associated with inflammation and scarring in the liver. This more serious form of the disease is termed metabolic dysfunction-associated steatohepatitis (MASH), formerly known as non-alcoholic steatohepatitis (NASH). MASH is associated with a much higher risk of liver fibrosis and cirrhosis than MASLD. Patients with MASH have increased risk for hepatocellular carcinoma. MASLD may progress to MASH with fibrosis cirrhosis and hepatocellular carcinoma.

The term “Metabolic dysfunction-Associated Steatotic Liver Disease” (MASLD), formerly known as “Non-Alcoholic Fatty Liver Disease” (NAFLD) refers to a common chronic liver disease characterized in part by a fatty liver condition with associated risk factors of obesity, metabolic syndrome, and insulin resistance. Both MASLD and MASH are often associated with obesity, diabetes mellitus and asymptomatic elevations of serum ALT and gamma-GT. Ultrasound monitoring can suggest the presence of a fatty infiltration of the liver; differentiation between MASLD and MASH, typically requires a liver biopsy.

The term “Metabolic dysfunction-associated Alcoholic Liver Disease (Met-ALD), refers to MASLD patients that consume greater amounts of alcohol per week (>140 g/week females and >210 g/week males).

The term “Primary Sclerosing Cholangitis” (PSC) refers to a chronic liver disease caused by progressive inflammation and scarring of the bile ducts of the liver. Scarring of the bile ducts can block the flow of bile, causing cholestasis. The inflammation can lead to liver cirrhosis, liver failure and liver cancer. Chronic biliary obstruction causes portal tract fibrosis and ultimately biliary cirrhosis and liver failure. The definitive treatment is liver transplantation. Indications for transplantation include recurrent bacterial cholangitis, jaundice refractory to medical and endoscopic treatment, decompensated cirrhosis and complications of portal hypertension (PHTN). PSC progresses through chronic inflammation, fibrosis/cirrhosis, altered portal circulation, portal hypertension and portal-systemic shunting to varices-ascites and encephalopathy. Altered portal flow is an indication of clinical complications.

The following abbreviations are employed in the present disclosure. 13C-CA=carbon-13-labeled cholate; AIC-Akaike Information Criterion; BMI=body mass index; CI=confidence interval; CLD=chronic liver disease; CM=compartmental model; d4-CA=deuterium-labeled cholate; DSI=disease severity index; ER=extraction ratio; HCV=hepatitis C virus; HFR=hepatic filtration rate; HR=hepatic reserve; ICC=intraclass correlation coefficient; IV=intravenous; LC-MS=liquid chromatography-mass spectrometry; LC-MS/MS=liquid chromatography-tandem mass spectrometry. MM=minimal model; MMvd=minimal model based on volume of distribution; MSE=mean squared error; NAFLD=nonalcoholic fatty liver disease; NASH=non-alcoholic steatohepatitis; SLD=steatotic liver disease; MetALD=metabolic dysfunction-associated alcoholic liver disease; MASLD=metabolic dysfunction-associated steatotic liver disease; MASH=metabolic dysfunction-associated steatohepatitis; TBV=total blood volume.

All patents, patent applications and publications referred to herein are incorporated by reference in their entirety.

The embodiments described in one aspect of the present disclosure are not limited to the aspect described. The embodiments may also be applied to a different aspect of the disclosure as long as the embodiments do not prevent these aspects of the disclosure from operating for its intended purpose.

### Compartmental Modeling Versus Non-Compartmental Analysis

Two main approaches are used herein to describe the pharmacokinetics (PK) of administered compounds: compartmental models and noncompartmental analysis.

Compartmental models divide the system or body into a series of one or more, two or more, or three or more, compartments of different volumes and are described by a series of kinetic equations simulating the transfer of flow from one compartment to another.

Compartmental models assume that each of the compartments is kinetically homogeneous and that the drug is instantaneously and evenly distributed throughout the compartment. The mathematics of compartmental modeling typically involve systems of first-order ordinary differential equations with constant coefficients. While some compartmental models are descriptive and achieve adequate fits to clearance data using two or three compartments, more realistic models which attempt to define underlying physiological mechanisms can be developed using multi-compartmental systems.

On the other hand, noncompartmental analysis, as the name implies, does not use assumptions about body compartments and is regarded as model independent. Noncompartmental analysis often uses relatively simple algebraic equations to estimate summary PK parameters. As a result, noncompartmental methods require fewer assumptions than model-based approaches and are generally simpler, faster, and cheaper to develop relative to compartmental models.

Previously, a noncompartmental analysis, hereafter referred as the minimal model (MM), was used to characterize intravenous (IV) clearance by exponential fits and oral clearance by a cubic spline fit (Everson, G. T., et al., Portal-systemic shunting in patients with fibrosis or cirrhosis due to chronic hepatitis C: the minimal model for measuring cholate clearances and shunt. Alimentary Pharmacology & Therapeutics, 2007. 26(3): p. 401-410).

More recently, the present inventors developed a compartmental model of the cholate SHUNT test which allowed determination of anatomic shunting and hepatic extraction, as well as improved the within individual reproducibility of SHUNT test measurements (McRae, M. P., et al., Compartmental model describing the physiological basis for the HepQuant SHUNT test. Translational Research, 2023; 252:53-63).

The present disclosure uses compartmental and/or noncompartmental methods to simultaneously estimate in an extracorporeal liver perfusion system (ECS), a native human liver, and an extracorporeal liver, one or more of distinguishable cholate systemic clearance, systemic hepatic filtration rate, portal clearance, portal hepatic filtration rate, SHUNT, STAT, DSI, Hepatic Reserve, and RCA20, and optionally compares the results to previous cholate liver function test measurements.

### Cholate Liver Function Test Versions

Various Cholate Liver Function Test versions are summarized below and are disclosed in US 2024/0175881 A1, which is incorporated herein by reference in its entirety.

Cholate SHUNT V1.0 liver function test involves the simultaneous intravenous and oral administration of two distinguishable cholates, for example, 13C-CA by IV and d4-CA orally, and six timed peripheral venous blood samples over 90 minutes. Cholate concentrations in serum are measured by LC/MS or LC/MS-MS, and a noncompartmental analysis fits IV and oral clearance curves to measurement data calculate the areas under the IV and oral curves (AUC_{IV}, AUC_{Oral}). (Everson et al. Portal-systemic shunting in patients with fibrosis or cirrhosis due to chronic hepatitis C: the minimal model for measuring cholate clearances and shunt. Aliment Pharmacol Ther. 2007; 26:401-10). The SHUNT V1.0 requires both oral and intravenous (IV) cholate doses and five or six peripheral venous blood samples taken over 90 minutes. The cholate SHUNT V1.0 liver function test (minimal model, MM=SHUNT V1.0) uses stable isotopes of cholate administered both intravenously (13C-CA) and orally (d4-CA) labeled cholates to quantify liver function and physiology from blood sampled at 5, 20, 45, 60, and 90 min for the distinguishable serum cholates. The 13C- and d4-CA concentrations are calculated based on cubic spline and exponential fits. Everson G T et al. Aliment. Pharmacol. Ther. 2007; 26:401-410. U.S. Pat. No. 8,778,299, Everson and US Pat. Appl. Pub. No. US 2021/0318274 A1, Everson et al. discloses SHUNT V1.0 methods.

Cholate SHUNT V1.1 liver function test eliminates the 5-minute sample from the calculations for the SHUNT V1.0. Instead, the 13C-CA is estimated from a total blood volume calculation (Lemmens et al., Estimating blood volume in obese and morbidly obese patients. Obes Surg. 2006; 16:773-6), and the 5-minute d4-CA is approximated by 15% of the 20-minute concentration. This modification was shown to significantly reduce the variability in measuring test parameters that depend on the systemic clearance. The SHUNT V1.1 cholate test includes oral and IV dosing but collection of only 4 blood samples. The intravenous 13C- and oral d4-CA concentrations except for the 5 min. data points and the 13C- and d4-CA concentrations are calculated based on cubic spline and exponential fits. Everson G T et al. Aliment. Pharmacol. Ther. 2007; 26:401-410. The SHUNT V1.1 cholate test is disclosed in US 2024/0175881 which is incorporated herein by reference in its entirety.

Here, V_{d} is first estimated by Equation 21, then the initial concentration is estimated by dividing the IV dose by V_{d} times body weight. This estimated initial concentration value is then used in the original SHUNT V1.0 equations to construct the IV clearance curve.

In some cases, the initial intravenous distinguishable cholate compound concentration at 0 minutes is estimated comprising estimating Vd (L per kg body weight) in the subject by equation 21:

$$TPV = {V}_{d} = \frac{0.07}{\sqrt{BMI / 22}} \cdot ( 1 - Hct ) , \qquad Eqn . 21$$

and
dividing the IV dose by Va times body weight (BW).

In some cases, the SHUNT V1.1 method for assessing liver function in a patient comprises
(a) receiving a plurality of blood or serum samples collected from the subject following oral administration of a dose of a first distinguishable cholate compound (doseₒᵣₐₗ) to the subject and simultaneous intravenous co-administration of a dose of a second distinguishable cholate compound (doseᵢᵥ) to the subject, wherein the samples had been collected over no more than 180 minutes after administration;
(b) quantifying the concentration of the first and the second distinguishable cholate compounds; and
(c) generating individual subject oral and intravenous clearance curves from the concentration of the first and second distinguishable cholate compounds comprising using a computer algorithm curve fitting to model oral and intravenous clearance curves; and computing the area under the individualized oral and intravenous clearance curves (AUCoral) and (AUCiv), respectively, in the subject, wherein the multiplicity of samples comprise blood or serum samples collected from the subject in at least 4 time points, and
wherein the generating individual intravenous clearance curve comprises estimating an initial intravenous distinguishable cholate compound concentration in the subject at 0 minutes comprising
estimating Vd (L per kg body weight) in the subject by equation 21:

$$TPV = {V}_{d} = \frac{0.07}{\sqrt{BMI / 22}} \cdot ( 1 - Hct ) ; \qquad Eqn . 21$$

dividing the IV dose by V_{d} times body weight (BW) to obtain the initial estimated initial intravenous distinguishable cholate concentration; and
constructing the intravenous distinguishable cholate clearance curve comprising the estimated initial intravenous concentration.

Cholate SHUNT V2.0 liver function test further simplifies by shortening the testing window and requiring only two blood draws, for example, at 20 and 60 minutes. Cholate SHUNT V2.0 uses a compartmental model (McRae et al., Compartmental model describing the physiological basis for the HepQuant SHUNT test. Transl Res. 2023; 252:53-63, and McRae M P, Kittelson J, Helmke S M, Everson G T. Within-individual reproducibility of a dual sample oral cholate challenge test (DuO) and simplified versions of the HepQuant SHUNT test. Clin Transl Sci. 2024; 17:e13786. doi:10.1111/cts.13786) to measure the portal cholate clearance and noncompartmental exponential fits to measure systemic cholate clearance. The SHUNT V2.0 cholate test is disclosed in US 2024/0175881 which is incorporated herein by reference in its entirety.

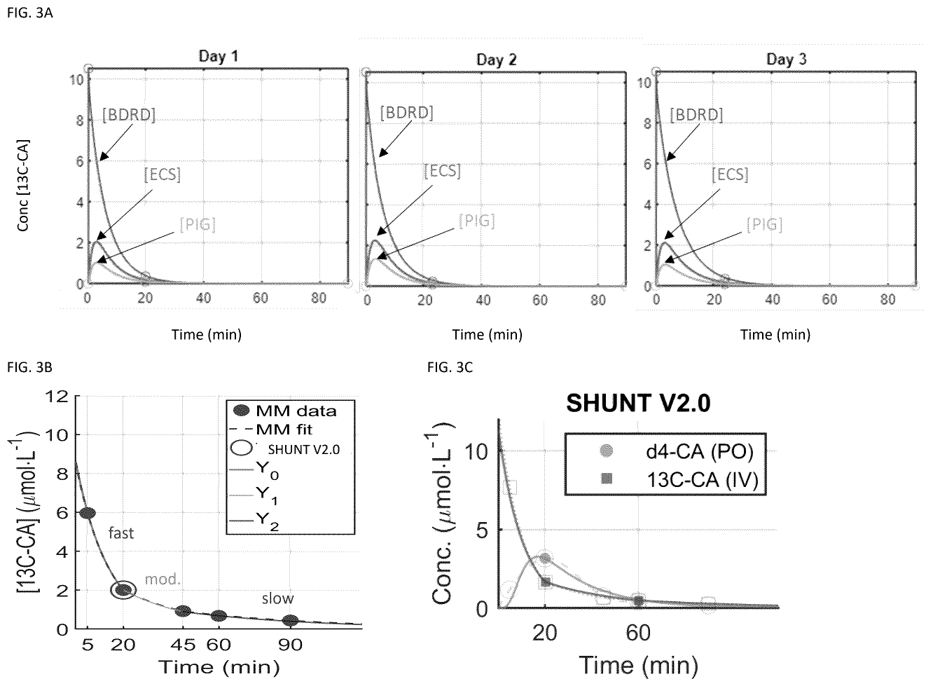

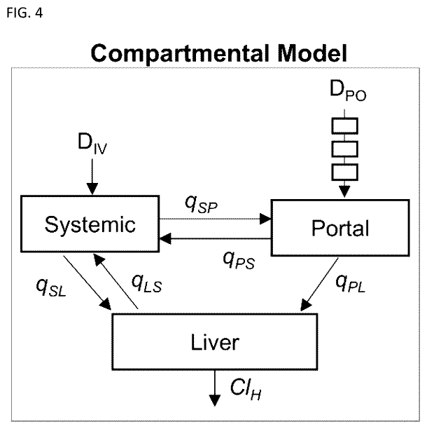

Briefly, the cholate SHUNT V2.0 (FIG. 3C) is a cholate liver function test that implements (1) a compartmental model of portal cholate clearance that uses assumptions of liver flow and physiology to predict oral clearance curves and (2) noncompartmental exponential fits to systemic cholate clearance. The compartmental model (Error! Reference source not found.) for measuring portal clearance describes the flow between systemic(S), portal (P), and liver (L) compartments represented by a system of first-order ordinary differential equations (Equations 13, 14, and 15), where q is the flow rate between compartments, V is the volume of the compartment, C is the concentration of d4-CA in the compartment, Cl_{H} is the hepatic clearance, and D_{PO,rate} is the rate of orally administered d4-CA entering the portal compartment.

$$\frac{{dC}_{S}}{dt} = \frac{1}{{V}_{S}} ( - [ {q}_{SP} + {q}_{SL} ] {C}_{S} + {q}_{LS} \cdot {C}_{L} + {q}_{PS} \cdot {C}_{P} ) \qquad Eqn . 13$$

$$\frac{{dC}_{P}}{dt} = \frac{1}{{V}_{P}} ( {D}_{PO , rate} + {q}_{SP} \cdot {C}_{S} - {q}_{PL} \cdot {C}_{P} - {q}_{PS} \cdot {C}_{P} ) \qquad Eqn . 14$$

$$\frac{{dC}_{L}}{dt} = \frac{1}{{V}_{L}} ( - [ {Cl}_{H} \cdot {V}_{L} + {q}_{LS} ] {C}_{L} + {q}_{PL} \cdot {C}_{P} + {q}_{SL} \cdot {C}_{S} ) \qquad Eqn . 15$$

The system of ordinary differential equations is solved numerically, and the concentration of d4-CA in the systemic compartment is integrated over 180 minutes to calculate the AUC_{Oral}.

A noncompartmental analysis method was developed involving the exponential fits of systemic cholate clearance using only a single time point, e.g., the 20 min. intravenous distinguishable (e.g., 13C-CA) concentration timepoint. The exponential fits split the curve into three sections or phases: fast, moderate, and slow (for example, 0 to 20 min., 20-45 min., and 45-180 min. for Y₀, Y₁, and Y₂, respectively) (FIG. 3B).

The noncompartmental analysis for measuring systemic clearance involves the exponential fits of systemic cholate clearance using at least one of the 20- and 60-minute 13C-CA concentrations. The exponential fits split the curve into three clearance phases: fast (Y₀), moderate (Y₁), and slow (Y₂). Equations 16, 17, and 18 define the systemic concentration of 13C-CA through time.

$${Y}_{0} = {C}_{0} \cdot {e}^{- {k}_{fast} \cdot t} \qquad Eqn . 16$$

$${Y}_{1} = \frac{{C}_{20}}{{e}^{- {k}_{mod} \cdot {T}_{20}}} \cdot {e}^{- {k}_{mod} \cdot t} \qquad Eqn . 17$$

$${Y}_{2} = \frac{{C}_{60}}{{e}^{- {k}_{slow} \cdot {T}_{60}}} \cdot {e}^{- {k}_{slow} \cdot t} \qquad Eqn . 18$$

Here, t is time (e.g., 0-20, 20-60, and 60-180 minutes for Y₀, Y₁, and Y₂, respectively), C₀ is the initial concentration of intravenous distinguishable cholate, e.g., 13C-CA, C₂₀ and C₆₀ are the measured 20- and 60-minute concentrations of intravenous distinguishable cholate, e.g., 13C-CA, and T₂₀ and T₆₀ are the actual collection times of the 20- and 60-minute blood sample. The systemic concentration of intravenous distinguishable cholate, e.g., 13C-CA, can then be integrated to calculate the AUC_{IV}.

In some cases, the fitting to systemic cholate clearance curve fast phase (Y₀) is calculated according to equation 16:

$${Y}_{0} = {C}_{0} \cdot {e}^{- {k}_{fast} \cdot t} , \qquad Eqn . 16$$

wherein
t=time (0 to 20 min);
C₀ is the initial concentration of intravenously administered distinguishable cholate compound,
C₂₀ is the measured 20-minute concentration of intravenously administered distinguishable cholate compound; and
k_{fast} is the rate of elimination in the fast phase estimated by equation 19:

$${k}_{fast} = \frac{ln ( {C}_{0} ) - ln ( {C}_{20} )}{{T}_{20}} , \qquad Eqn . 19$$

optionally wherein
T₂₀ is the actual time recorded for the 20-minute sample; and
C₀ is estimated according to equation 20;

$${C}_{0} = \frac{{D}_{IV}}{{V}_{d} \cdot BW} , \qquad Eqn . 20$$

wherein
D_{IV} is the intravenous dose of third distinguishable cholate;
BW is subject body weight (kg); and
Vd is the volume of distribution (V_{d}) in L per kg body weight, calculated according to equation 21:

$$TPV = {V}_{d} = \frac{0.07}{\sqrt{BMI / 22}} \cdot ( 1 - Hct ) , \qquad Eqn . 21$$

wherein TPV is total plasma volume, BMI is body mass index, and Hct is hematocrit in the subject.

In some cases, the fitting to systemic cholate clearance curve moderate phase (Y₁) is calculated according to equation 22:

$${Y}_{1} = \frac{{C}_{20}}{{e}^{- {k}_{mod} \cdot 20}} \cdot {e}^{- {k}_{mod} \cdot t} , \qquad Eqn . 22$$

wherein
t=time (20-45 min);
k_{mod} is the rate of elimination in the moderate phase estimated by equation 23:

$${k}_{mod} = - \frac{ln ( {C}_{60} ) - ln ( {C}_{20} )}{{T}_{60} - {T}_{20}} , \qquad Eqn . 23$$

C₆₀ is the concentration at 60 minutes;
C₂₀ is the concentration at 20 minutes;
T₆₀ is the actual time recorded for the 60-minute sample; and
T₂₀ is the actual time recorded for the 20-minute sample.

In some cases, the fitting to systemic cholate clearance curve slow phase (Y₂) is calculated according to equation 24:

$${Y}_{2} = \frac{{C}_{45}}{{e}^{- {k}_{slow} \cdot 45}} \cdot {e}^{- {k}_{slow} \cdot t} , \qquad Eqn . 24$$

wherein
t=time (45-180 min);
C₄₅ is the estimated 45-minute concentration of intravenously administered distinguishable cholate; and
k_{slow} is the rate of elimination in the slow phase estimated by a mean value from a multiplicity of CLD patients, optionally wherein k_{slow} is 0.018 min⁻¹.

In some cases, the areas under each of the three exponential curve fits (fast, moderate, and slow) are calculated by trapezoidal numerical integration and summed to estimate the AUC_{IV}.

DuO cholate liver function test is an oral-only test, for example, involving only one oral dose at 0 minutes and two blood samples collected at 20 and 60 minutes. The IV clearance is derived rather than measured, and the derived IV concentrations are then used in the same noncompartmental analysis as SHUNT V2.0. DuO is described in US 2024/0175881 which is incorporated herein by reference.

The cholate DuO liver function test, also known as Dual Sample Oral Cholate Challenge Test (also known as “DuO”, “DUO”, “DuO test”, “DuO cholate test”, “HepQuant DUO”, “HepQuant DuO”) is a compartmental model of portal cholate clearance that uses assumptions of liver flow and physiology to predict oral clearance curves comprising measuring only two (e.g., 20 min. and 60 min.) orally administered distinguishable cholate (e.g., d4-CA) concentration timepoints (i.e., DuO liver function test v1.0). The DuO v1.0 test comprises administration of 1 oral dose of a distinguishable cholate (e.g., d4-CA at 0 min.), and collection of 2 blood draws at first and second time points (e.g., 20 and 60 min.). The DuO v2.0 test comprises administration of 2 oral doses of first and second distinguishable cholates, respectively, at first and second time points (e.g., d4-CA at 0 min., 13C-CA at 40 min.), and collection of 1 blood draw at a single time point (e.g., 60 min.). The DuO test quantifies portal HFR and estimates systemic HFR, DSI, SHUNT %, and Hepatic Reserve (HR) using only oral dose(s). The DuO dual oral cholate clearance tests do not require measurement of an intravenously administered distinguishable cholate.

Cholate STAT liver function test is the simplest test method which involves one oral cholate dose and one blood sample at, e.g., 60 minutes. The value of the STAT test may be reported as either the STAT score (e.g., the d4-CA concentration adjusted to 75 kg body weight), the portal HFR estimated by STAT, or DSI estimated by STAT. The cholate STAT test is described in US 2024/0175881, which is incorporated herein by reference.

### Test Parameters

The various cholate liver function tests return a series of test parameters also known as hepatic indices which have been previously associated with liver function and disease/health status. This following cholate test parameters or hepatic indices may be determined:

Portal hepatic filtration rate (HFRP) is the portal clearance adjusted for body weight (units mL min⁻¹ kg⁻¹) and was calculated by Equation 25:

$${HFR}_{P} = \frac{{D}_{PO}}{{AUC}_{Oral} \cdot BW} , \qquad Eqn . 25$$

where D_{PO} is the administered oral cholate dose of distinguishable cholate, and BW is the subject body weight.

Systemic hepatic filtration rate (HFRs) is the measured systemic clearance adjusted for body weight (units mL min⁻¹ kg⁻¹) and was calculated by Equation 26:

$${HFR}_{S} = \frac{{D}_{IV}}{{AUC}_{IV} \cdot BW} , \qquad Eqn . 26$$

wherein D_{IV} is the administered intravenous dose of distinguishable cholate, and BW is the subject body weight.

Disease Severity Index (DSI) is a score between 0 and 50 which indicates overall liver function comprising both portal and systemic HFR. DSI is associated with fibrosis stage and clinical stages of cirrhosis and was calculated by Equation 27:

$$DSI = A \cdot \sqrt{{( ln [ \frac{{HFR}_{P , m ax}}{{HFR}_{P}} ] )}^{2} + {( ln [ \frac{{HFR}_{S , m ax}}{{HFR}_{S}} ] )}^{2}} , \qquad Eqn . 27$$

where A is a scaling factor and HFR_{P,max} and HFR_{S,max} are the upper limits of clearance for healthy controls.

SHUNT %, or shunt fraction, is the estimated absolute bioavailability of the oral d4-CA dose in systemic circulation estimated by Equation 28. SHUNT % is a direct measurement of the first pass hepatic extraction of cholate which is influenced by portal blood flow and portal-systemic shunt.

$$SHUNT \% = \frac{{AUC}_{Oral} \cdot {D}_{IV}}{{AUC}_{IV} \cdot {D}_{PO}} , \qquad Eqn . 28$$

wherein AUCₒᵣₐₗ is the area under the curve of the serum concentrations of the orally administered distinguishable cholate compound, D_{IV} is the dose of the intravenously administered distinguishable cholate compound, AUCiv is the area under the curve of the intravenously administered distinguishable cholate compound, and D_{PO} is the dose of the orally administered distinguishable cholate compound.

Hepatic Reserve (HR) represents an individual's overall hepatic health relative to lean healthy controls, with values between 100 (healthy) and 0 (severely impaired). HR can be calculated by Equation 29:

$$HR = 100 - A \cdot \sqrt{{( ln [ \frac{{HFR}_{P , lean}}{{HFR}_{P}} ] )}^{2} + {( ln [ \frac{{HFR}_{S , lean}}{{HFR}_{S}} ] )}^{2}} , \qquad Eqn . 29$$

where A is a scaling factor and HFR_{P,lean} and HFR_{S,lean} are mean HFRs minus one standard deviation from a population of lean controls.

RCA20 represents the amount of the intravenously administered distinguishable compound, for example, a distinguishable cholate compound such as 13C-CA, that remains in the circulation 20 minutes after the intravenous injection. The formula for RCA20 may be calculated by Equation 30:

$$RCA 20 = ( 1 - ( {[ {}^{13} C CA ]}_{t = 0} - ( {[ {}^{13} C CA ]}_{t = 20} ) / {[ {}^{13} C CA ]}_{t = 0} ) \times 100 \% , , \qquad Eqn . 30$$

wherein RCA20 represents the amount of the intravenously administered 13C-CA that remains in the circulation 20 minutes after the intravenous injection. [¹³C CA]ₜ₋₀ is determined from Ln/linear regression of [13C-CA] versus time.

Any appropriate analysis method known in the art may be employed for quantification of the distinguishable cholate compounds in blood or serum samples. For example, detection and quantification of the distinguishable cholate compound in the sample may comprise high performance liquid chromatography (HPLC), HPLC-diode-array detection (HPLC-DAD), HPLC-fluorescence, ultra-performance liquid chromatography (UPLC), mass spectrometry (MS), GC-MS, LC-MS, LC-MS/MS, surface-enhanced Raman spectroscopy (SERS), immunoassays, for example, using isolated antibodies, monoclonal antibodies, or antigen-binding fragments thereof, single domain antibodies, aptamers, and the like. Methods for detection and quantification of distinguishable cholates are described in, for example, US20210318274, which is incorporated herein by reference in its entirety.

The blood or serum sample for use in the present methods may be collected from an ECS and/or from a subject by any known method in the art. For example, see WHO guidelines on drawing blood: best practices in phlebotomy, World Health Organization, 2010, Geneva, Switzerland or BP-EIA: Collecting, processing, and handling venous, capillary, and blood spot samples, PATH, 2005. For example, venipuncture using needle and syringe or indwelling catheter, arterial blood sampling, pediatric or neonatal blood sampling, or capillary sampling may be employed. The choice of site and procedure may depend on the volume of blood needed for the procedure and laboratory test to be done. For example, a venous site, finger-prick or heel-prick, also known as capillary sampling or skin puncture, may be employed.

Whole blood samples may be obtained by any acceptable technique, for example, at any point in the system, or from the patient by venipuncture, collected in anticoagulant-containing vacutainer tubes, and refrigerated during storage and shipment. Blood samples can be further processed into different fractions. The blood or serum samples may be peripheral blood samples. The sample may be a transcutaneous blood sample. Various commercial devices are available for obtaining transcutaneous samples such as, for example, single-use blood lancing devices intended for obtaining microliter capillary whole blood samples (e.g., Tasso, Inc., Seattle, WA). The sample may be a capillary, venous blood, or serum sample.

The volume of the blood or serum sample may be from 10 microliters to 0.5 milliliters, or more. In some cases, the volume of the blood or serum sample may be from 20 microliters to 0.4 milliliters, or 30 microliters to 0.3 milliliters. In some cases, the blood or serum sample volume can be as low as, for example, 10, 20 or 30 microliters or more. Any appropriate form of sample extraction may be employed, as known in the art. Analysis may be performed according to any appropriate means, for example, LC-MS/MS, for example, as described in US 2021/0318274 A1.

### Compartmental Analysis of Extracorporeal Liver Perfusion Systems

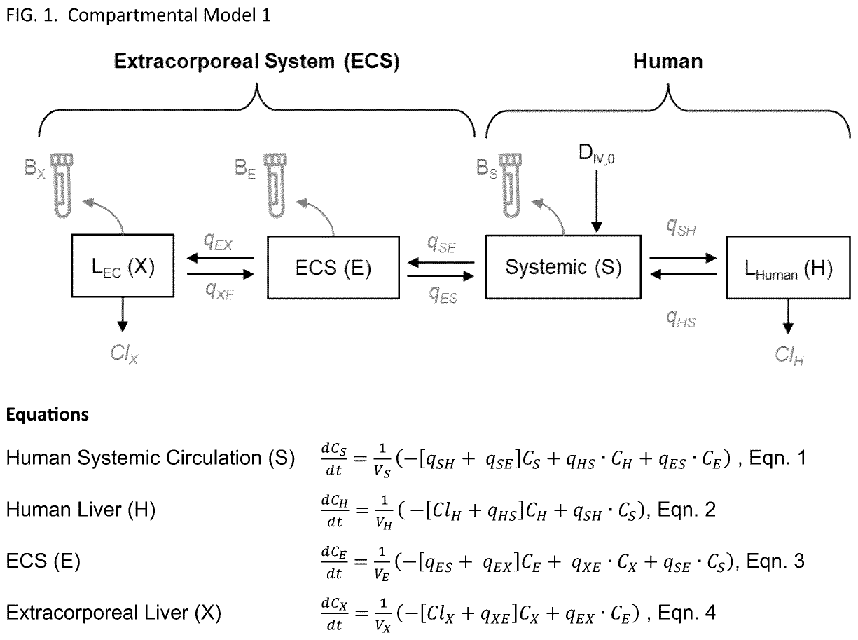

A method for compartmental analysis of the distinguishable cholate compound assay in extracorporeal liver perfusion systems (ECS) is provided (FIG. 1). FIG. 1 shows a schematic of an inventive compartmental model of simultaneous clearance of intravenously administered distinguishable cholate by a human patient liver and an extracorporeal liver in an extracorporeal liver perfusion system (ECS).

In the ECS compartmental model 1, a system of ordinary differential equations 1-4 as shown in FIG. 1 is solved to estimate the liver clearance parameters in the human liver, Cl_{H}, and in the extracorporeal (EC) liver, Cl_{X}, effectively distinguishing the abilities of the human and EC livers to extract cholate and, thus, measure liver function.

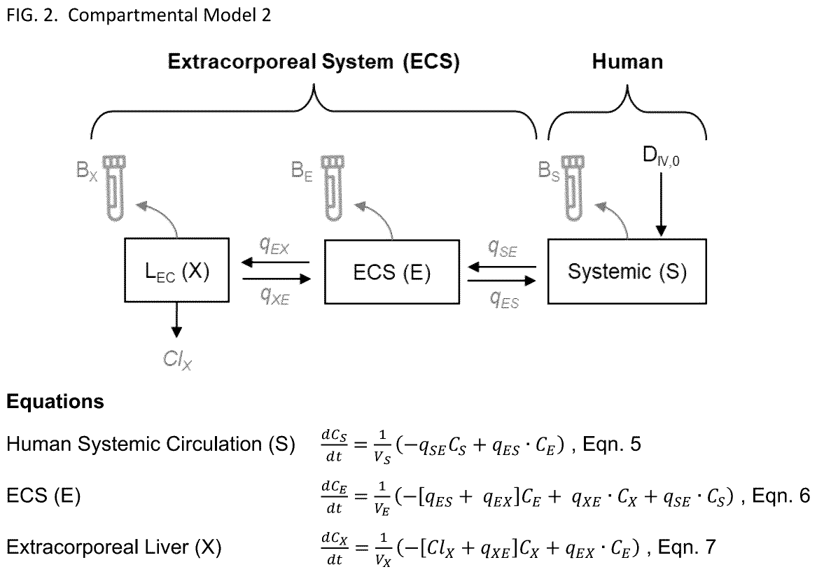

A variant of the compartmental analysis may also involve a human patient with their liver removed, with cholate extraction performed entirely by the extracorporeal (EC) liver (FIG. 2). In the ECS compartmental model 2, a system of ordinary differential equations 5-7 as shown in FIG. 2 is solved to estimate the liver clearance parameters in the extracorporeal (EC) liver, Cl_{X}, to measure the abilities of the EC liver to extract cholate and, thus, measure EC liver function.

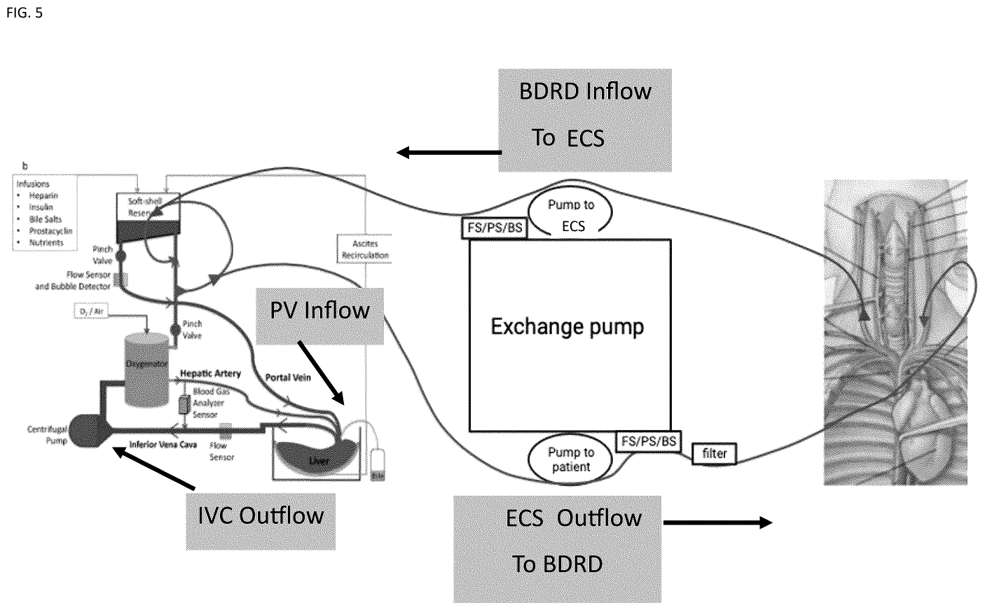

Extracorporeal liver perfusion systems (ECS) are commercially available. For example, one portable commercially available ECS is the Organox METRA®, OrganOx Limited, Oxford, UK. During normothermic machine perfusion, the donor liver is continuously perfused with oxygenated blood, medications, and nutrients at normal body temperature and near physiological pressures and flows. The ECS comprises an oxygenator, a heater, a pump, a reservoir, and medicines and nutrition. Oxygen is concentrated from ambient air and supplied on demand to the oxygenator. The blood is also warmed by an on-board heater. Blood is drawn from the liver by a centrifugal pump, which automatically varies in speed according to changes in blood pressure. Warm oxygenated blood is stored in a soft shell reservoir and supplied to the liver under near physiological pressure. Medicines and nutrition are delivered automatically through perfusion to minimize operator hands-on time. Infusions may include heparin, insulin, bile salts, prostacyclin, and nutrients. A schematic diagram of one prior art ECS; the Organox METRA® exchange system design is shown in FIG. 5.

Extracorporeal (EC) livers are known in the art. The EC liver can be a genetically engineered porcine liver. In some cases, the genetically engineered donor pigs can include (i) knock out of three genes involved in synthesis of glycan antigens implicated in hyperacute rejection, (ii) insertion of several human transgenes involved in regulation of several pathways that modulate rejection: inflammation, innate immunity, coagulation, and complement, and (iii) inactivation of the endogenous retroviruses in the porcine genome. Anand et al., 2023, Design and testing of a humanized porcine donor for xenotransplantation, Nature, 622, 12 October, 393, doi.org/10.1038/s41586-023-06594-4.

The present ECS compartmental model 1 was adapted from a previous compartmental model¹ of a cholate challenge test.^{2.3} The compartmental analysis of the ECS is summarized below.

The ECS compartmental model 1 as shown in FIG. 1 contains 4 theoretical compartments:
Human systemic circulation (S);
Human patient liver (H);
Extracorporeal liver perfusion system (E); and
Extracorporeal liver (X).

### Compartment Volumes

Total blood volume (TBV) is calculated by TBV=(0.07*wt)/sqrt(bmi/22) which accounts for the nonlinear relationship between blood volume and body mass index (bmi, kg/m²) across the entire range of body weights (wt) including obese (BMI 30-40) and morbidly obese (BMI>40) subjects.⁴ In this equation, 22 is the BMI value corresponding to ideal body weight, and 0.07 is the indexed blood volume (in L/kg) for a subject with a BMI of 22.

To calculate the volume of the human liver (V_{H}) compartment, liver volumes corrected for body weight from 13 studies of ultrasound and computerized tomography scans⁵ are averaged (22.46±1.98 mL/kg) and multiplied by body weight and liver tissue density (d) to obtain liver weight (w_{L}). Liver tissue density of 1.06 g/mL is derived from the average of two studies estimating liver densities of 1.04 g/mL and 1.08 g/mL.^{6,7} Hepatic blood volume ranges from 25 to 30 mL per 100 g liver weight.^{8,9} Using the average of this range (0.275 mL/g), the volume of the human liver compartment is estimated by V_{H}=(0.275*22.46*wt*dL)/1000.

Volume of the human systemic circulation (V_{S}) is calculated by TBV minus the liver volume, V_{H}.

Volume of the ECS (V_{E}) is provided by the manufacturer information.

Volume of the EC liver (V_{X}) is estimated using the EC liver weight multiplied by the average hepatic blood volume per liver weight, same as for the human liver above.

### Flow Rates

Flow rates between the compartments are either measured directly or derived.

Total blood flow into the human patient liver (q_{SH}) is the combined flow of hepatic arterial and portal venous inflows. Using the previous estimate for liver weight, the total hepatic inflow rate is approximately 1 L/min/kg liver wet weight.^{8,10}

Unextracted distinguishable cholate compound, such as 13C-CA, returns to systemic circulation (q_{HS}) at the same flow rate as q_{SH}.

The inflow to the ECS (q_{SE}) is measured, and the blood outflow from the ECS back to the patient (q_{ES}) is assumed to be equal to the inflow, q_{SE}.

The total blood inflow to the EC liver from the ECS (q_{EX}) is the sum of the measured portal venous blood inflow (q_{EX,PV}) and measured hepatic arterial inflow (q_{EX,HA}) to the EC liver.

The IVC blood outflow from the EC liver back to the ECS (q_{XE}) is also measured and should equal the EC liver inflow, q_{EX}.

### Dosing

An intravenous (IV) dose (D_{IV}) of a first distinguishable cholate compound, such as 13C-labelled cholic acid (13C-CA), is injected into the systemic circulation of the human patient(S). The initial concentration of the first distinguishable cholate compound, such as 13C-CA, in systemic circulation (C₀) immediately after administration is the dose, D_{IV}, divided by the systemic compartment volume, V_{S}.

An oral dose (Dₒᵣₐₗ) of a second distinguishable cholate compound, such as d4-labelled cholic acid, is optionally administered to the human patient orally or through a nasogastric (NG) tube.

### Clearance

The human patient liver (H) extracts the first distinguishable cholate compound, such as 13C-CA, from the blood with clearance Cl_{H}.

The EC liver (X) extracts the first distinguishable cholate compound, such as 13C-CA, from the blood with clearance Cl_{X}.

Blood samples are collected from various compartments, and concentrations (C) of the first distinguishable cholate compound, such as 13C-CA, at various timepoints are determined and used to fit the clearance curves for parameter estimation. The blood samples can include blood samples from the systemic circulation of the human patient (B_{S}); blood samples from the ECS (B_{E}); and blood samples from the IVC outflow of the EC liver (B_{X}).

An example of the ECS compartmental model 1 analysis was completed for a first brain-dead research donor (BDRD) and an extracorporeal porcine liver using the parameters listed in Table 1.

TABLE 1
Compartmental model parameters for a brain-dead
research donor connected to an extracorporeal liver
perfusion system supporting a porcine liver.

| Description | Parameter | Value | Units |
|---|---|---|---|
| Patient body weight | wt | 83.9 | kg |
| Patient height | ht | 172.7 | cm |
| Patient BMI | bmi | 28.1 | kg/m² |
| Patient hematocrit | Hct | 0.254 | — |
| Weighted average of liver | — | 22.456 | mL/kg |
| volume per kg body weight | | | |
| Liver density | — | 1.060 | g/mL |
| Patient estimated liver weight | — | 1997 | g |
| EC liver weight | — | 1301 | g |
| Hepatic blood volume per 100 g | — | 0.275 | L per 100 g |
| liver weight | | | liver weight |
| Total blood volume of patient | TBV | 5.194 | L |
| Systemic compartment blood | V_{S} | 4.645 | L |
| volume | | | |
| Human patient liver blood | V_{H} | 0.549 | L |
| volume | | | |
| Extracorporeal system blood | V_{E} | 1.500 | L |
| volume | | | |
| Extracorporeal liver blood | V_{X} | 0.358 | L |
| volume | | | |
| Total hepatic blood flow per | — | 1.000 | L/min/kg liver |
| kg liver weight | | | wet weight |
| Inflow to human liver (hepatic | q_{SH} | 1.997 | L/min |
| arterial and portal venous) | | | |
| Outflow from human liver back | q_{HS} | 1.997 | L/min |
| to systemic circulation | | | |
| Inflow from patient systemic | q_{SE} | 0.35 | L/min |
| circulation to ECS | | | |
| Outflow from ECS to patient | q_{ES} | 0.35 | L/min |
| systemic circulation | | | |
| IVC blood outflow from EC | q_{XE} | 1.40 | L/min |
| liver back to ECS | | | |
| PV blood inflow to EC liver | q_{EX, PV} | 1.20 | L/min |
| from ECS | | | |
| Hepatic arterial inflow to EC | q_{EX, HA} | 0.2 | L/min |
| liver from ECS | | | |
| Total blood inflow to EC liver | q_{EX} | 1.4 | L/min |
| from ECS | | | |
| Clearance of the human liver | Cl_{H} | solve | L/min |
| Clearance of the EC liver | Cl_{X} | solve | L/min |
| Dose of intravenously- | D_{IV} | 49 | μmol |
| administered 13C-CA | | | |
| Initial estimate for human liver | Cl_{H, init} | 0.857 | L/min |
| clearance prior to ECS | | | |
| Initial estimate for EC liver | Cl_{X, init} | 1.223 | L/min |
| clearance prior to ECS | | | |
| Initial concentration of 13C-CA | C₀ | 10.55 | μM |
| in systemic circulation (S) | | | |

A system of ordinary differential equations 1-4 (Eqns. 1-4 as illustrated in FIG. 1) was developed and can be solved to estimate the liver clearance parameters, CH and Cl_{X}, effectively distinguishing the abilities of the human and EC livers to extract cholate and, thus, measure liver function.

### Human Systemic Circulation(S), Eqn. 1:

$$\frac{{dC}_{S}}{dt} = \frac{1}{{V}_{S}} ( - [ {q}_{SH} + {q}_{SE} ] {C}_{S} + {q}_{HS} \cdot {C}_{H} + {q}_{ES} \cdot {C}_{E} ) , \qquad Eqn . 1 ;$$

### Human Liver (H), Eqn. 2:

$$\frac{{dC}_{H}}{dt} = \frac{1}{{V}_{H}} ( - [ {Cl}_{H} + {q}_{HS} ] {C}_{H} + {q}_{SH} \cdot {C}_{S} ) , \qquad Eqn . 2 ;$$

### ECS (E), Eqn. 3:

$$\frac{{dC}_{E}}{dt} = \frac{1}{{V}_{E}} ( - [ {q}_{ES} + {q}_{EX} ] {C}_{E} + {q}_{XE} \cdot {C}_{X} + {q}_{SE} \cdot {C}_{S} ) , \qquad Eqn . 3 ;$$

and

### Extracorporeal Liver (X), Eqn. 4:

$$\frac{{dC}_{X}}{dt} = \frac{1}{{V}_{X}} ( - [ {Cl}_{X} + {q}_{XE} ] {C}_{X} + {q}_{EX} \cdot {C}_{E} ) , \qquad Eqn . 4 ,$$

wherein

V is the volume (L) in each of the patient systemic circulation (V_{S}), patient human liver (V_{H}), ECS (VE), and extracorporeal liver (V_{X}) compartments; C is the blood concentration (mM) of first distinguishable cholate in each of the patient systemic circulation (C_{S}), patient human liver (C_{H}), ECS (C_{E}), and extracorporeal liver (C_{X}) compartments; q is the blood flow rate between compartments (L/min); Cl_{H} is the hepatic clearance (L/min) of the human liver, and Cl_{X} is the hepatic clearance (L/min) of the extracorporeal liver.

In some cases, the patient or BDRD can undergo the cholate assay testing before connecting the ECS to define the initial value of clearance in the human liver (Cl_{H,init}). Likewise, the EC liver donor pig can also undergo cholate assay testing prior to the ECS to define the initial value of clearance in the pig liver (Cl_{X,init}). These initial values can be used in nonlinear least-squares regression to solve for the unknown clearance parameters, Cl_{H} and Cl_{X}, when both livers are connected to the ECS.

In one example, using a first BRRD (BDRD1), the liver clearances were estimated after 1, 2, and 3 days of perfusion on the ECS. Blood samples were collected and blood flows were measured from the BDRD, the ECS, and the IVC outflow of the extracorporeal pig liver after 20 and 60 minutes following intravenous injection of 13C-CA.^{2,3} Table 2 shows ECS compartmental model 1-estimated clearances for the human (Cl_{H}) and pig (Cl_{X}) livers for over 3 days on the extracorporeal liver perfusion system.

TABLE 2
ECS Compartmental model 1-estimated clearances
for the human (Cl_{H}) and pig (Cl_{X}) livers for over
3 days on the extracorporeal liver perfusion system.
Unpooled Parameter Estimates

| | |
|---|---|
| CL_{H} (L/min) | CL_{X} (L/min) |

| Group | Estimate | SE | Estimate | SE |
|---|---|---|---|---|
| Pre | 0.8572 | 0.0990 | NA | NA |
| Day 1 | 0.8728 | 0.0202 | 1.3840 | 0.1328 |
| Day 2 | 0.8243 | 0.0218 | 0.9348 | 0.0704 |
| Day 3 | 0.5906 | 0.0288 | 1.4347 | 0.2980 |

Statistics*

| | | | Log | | | |
|---|---|---|---|---|---|---|
| Group | AIC | BIC | Likelihood | DFE | MSE | SSE |
| Pre | −1.2538 | −3.0566 | 2.6269 | 1 | 0.0305 | 0.0305 |
| Day 1 | −102.075 | −100.295 | 53.038 | 16 | 0.0002 | 0.0029 |
| Day 2 | −51.804 | −51.409 | 27.902 | 7 | 0.0002 | 0.0011 |
| Day 3 | −43.601 | −43.207 | 23.801 | 7 | 0.0004 | 0.0027 |

*AIC, Akaike information criterion; BIC, Bayesian information criterion; DFE, degrees of freedom for error; MSE, mean squared error; SSE, sum of squared errors.

The ECS compartmental model 1 analyses for days 1, 2, and 3 in BDRD 1 are shown in FIG. 3A. Table 2 shows unpooled parameter estimates for the clearances of the human liver and the pig liver across 3 days. In this example, the results suggest that after 3 days on the ECS, the function of the pig liver was maintained while the function of the human liver declined.

An alternative ECS compartmental model 2 is provided of the clearance of intravenously administered cholate compounds by an extracorporeal liver in an extracorporeal liver perfusion system (ECS) after having removed the human patient's liver, as illustrated in FIG. 2.

A system of ordinary differential equations 5-7 (Eqns. 5-7 as illustrated in FIG. 2) was developed and can be solved to estimate the liver clearance parameter, Clx, to effectively determine the ability of the EC liver to extract cholate and, thus, measure EC liver function in support of the human patient.

### Human Systemic Circulation(S):

$$\frac{{dC}_{S}}{dt} = \frac{1}{{V}_{S}} ( - {q}_{SE} {C}_{S} + {q}_{ES} \cdot {C}_{E} ) , \qquad Eqn . 5 ;$$

### ECS (E):

$$\frac{{dC}_{E}}{dt} = \frac{1}{{V}_{E}} ( - [ {q}_{ES} + {q}_{EX} ] {C}_{E} + {q}_{XE} \cdot {C}_{X} + {q}_{SE} \cdot {C}_{S} ) , \qquad Eqn . 6 ;$$

and

### Extracorporeal Liver (X):

$$\frac{{dC}_{X}}{dt} = \frac{1}{{V}_{X}} ( - [ {Cl}_{X} + {q}_{XE} ] {C}_{X} + {q}_{EX} \cdot {C}_{E} ) , \qquad Eqn . 7 ,$$

wherein
V is the volume (L) in each of the patient or BDRD systemic circulation (V_{S}), the ECS (V_{E}), and the extracorporeal liver (V_{X}) compartments; C is the blood concentration (mM) of first distinguishable cholate in each of the patient systemic circulation (C_{S}), ECS (C_{E}), and extracorporeal liver (C_{X}) compartments; q is the blood flow rate between compartments (L/min); and Cl_{X} is the hepatic clearance (L/min) of the extracorporeal liver. The blood flow rate between compartments (L/min) q_{SE} is inflow from patient systemic circulation to ECS, q_{ES} is outflow from ECS to patient systemic circulation, q_{XE} is inferior vena cava (IVC) blood outflow from EC liver back to ECS, and q_{EX} is total blood inflow to EC liver from ECS.

In a prophetic example of the ECS compartmental model 2, using a human patient or a BRRD, the extracorporeal liver clearances can be estimated after one or more days, two or more days, e.g., 1, 2, and/or 3 days of perfusion on the ECS. Blood samples can be collected and blood flows measured from the patient or BDRD, the ECS, and the IVC outflow of the extracorporeal pig liver after one or more or two or more time points, e.g. 20 and 60 minutes, following intravenous injection of first distinguishable cholate (e.g., 13C-CA).

Eqns. 5-7 can then be solved to estimate the liver clearance parameter, Cl_{X}, to effectively determine the ability of the EC liver to extract cholate and, thus, measure EC liver function in the ECS in support of the human patient.

### Non-Compartmental Analysis of Extracorporeal Liver Perfusion Systems

An alternative ECS non-compartmental liver function analysis model was developed.

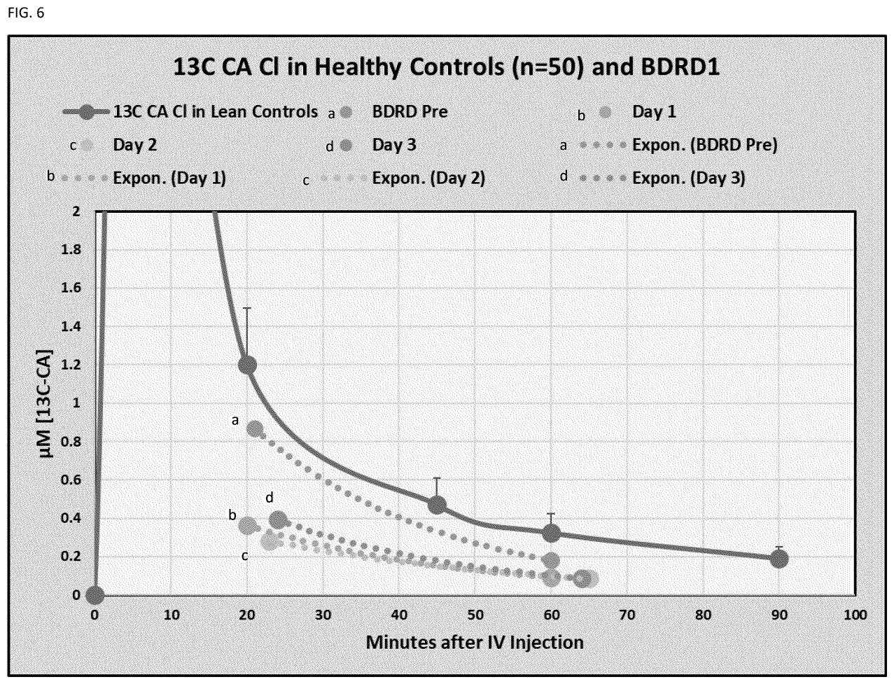

FIG. 6 shows a graph of intravenously administered distinguishable cholate concentration [13C-CA] in blood or serum samples of healthy controls and BRDR1 blood or serum samples. The clearance of [13C-CA] injected intravenously in healthy controls (n=50) is shown by the solid curve. Each point and whisker represents the mean intravenously administered distinguishable cholate concentration [13C-CA] and 1 SD for 50 measurements in healthy control persons. The dotted curve (a) markers are the raw 20 and 60 minute concentrations of [13C-CA] in the BDRD1, pre-ECS. Although lower than the mean, they are within 95% CI of normal. The dotted curves at (b) day 1, (c) day 2, and (d) day 3 represent the results for days 1, 2, and 3 and are markedly lower than normal and lower than the BDRD1, pre-ECS. This is consistent with the ECS functioning to clear cholate.

A non-compartmental analysis was developed for calculating the total clearance of an intravenously administered distinguishable cholate compound, such as 13C-cholate, in the human-ECS system and relative clearances of the native human liver and extracorporeal liver.

The total systemic clearance (Cl_{Tot}) of the human-ECS can be measured by the intravenous clearance of cholate (refer to SHUNT V1.0, V1.1, or V2.0 method). In some cases, the total systemic clearance (Cl_{Tot}) of the human-ECS can be measured by the cholate SHUNT V2.0 method using 20 and/or 60 minute [13C-CA] from blood or serum samples, for example, collected from BDRD1 inflow.

Concentrations, C, and flow rates, q, between components can be directly measured (refer to FIG. 1 and Table 1 for definitions).

The extraction efficiency of the ECS (EE) is time-dependent and related to the concentration and flow rates in/out of the ECS.

Given the concentration dependence on extraction (i.e., greater extraction efficiency at higher concentrations), the extraction efficiencies can be calculated at t=20 minutes according to the Equation 8:

$${E}_{E} ( t ) = \frac{{C}_{S} ( t ) \cdot {q}_{SE} ( t ) - {C}_{E} ( t ) \cdot {q}_{ES} ( t )}{{C}_{S} ( t ) \cdot {q}_{SE} ( t )} , \qquad Eqn . 8.$$

Similarly, the extraction efficiency of the extracorporeal liver (E_{X}) can be calculated by Equation 9:

$${E}_{X} ( t ) = \frac{{C}_{E} ( t ) \cdot {q}_{EX} ( t ) - {C}_{X} ( t ) \cdot {q}_{XE} ( t )}{{C}_{E} ( t ) \cdot {q}_{EX} ( t )} , \qquad Eqn . 9$$

The general formula for clearance is flow rate times extraction efficiency.

The clearance of the ECS (Cl_{E}) can be calculated as the flow rate through the ECS times extraction of the ECS, as shown in Equation 10:

$${Cl}_{E} = {q}_{SE} \cdot {E}_{E} , \qquad Eqn . 10.$$

Similarly, the clearance of the extracorporeal liver (Cl_{X}) can be calculated as the flow rate through the liver times the extraction of the liver, as shown in Equation 11:

$${Cl}_{X} = {q}_{XE} \cdot {E}_{X} , \qquad Eqn . 11.$$

The clearance of the human liver can be defined as the total clearance minus the clearance of the ECS, as shown in Equation 12:

$${Cl}_{H} = {Cl}_{Tot} - {Cl}_{E} , \qquad Eqn . 12.$$

Systemic hepatic filtration rate (HFR) can be defined as clearance normalized by body weight (units mL/min/kg).

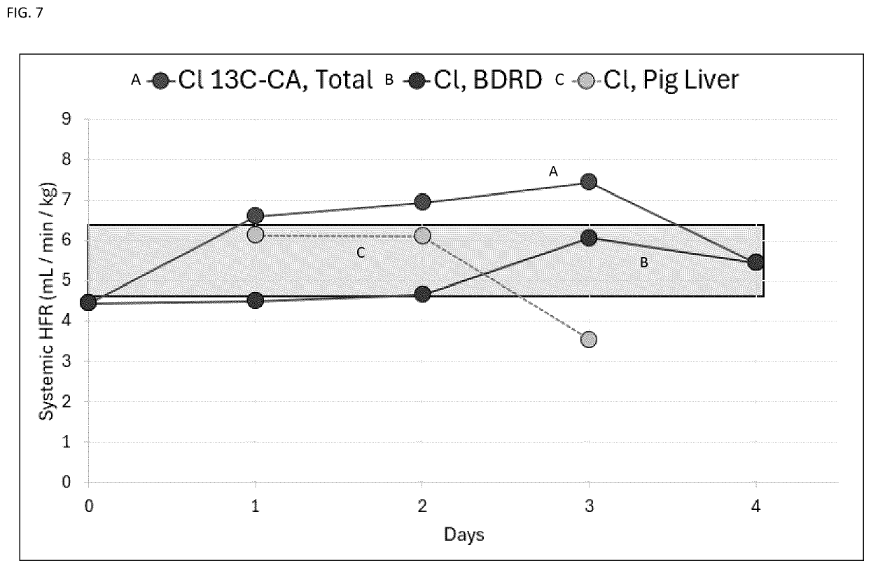

An example of the systemic clearances plotted as systemic HFR for a brain-dead research donor (BDRD3) connected to the ECS with a pig liver is shown in FIG. 7. In this example, systemic HFR of the pig liver declines at day 3. However, systemic HFR in the BDRD3 native human liver improves at day 3 into the normal range and remains in the normal range at day 4.

### CLAUSES

Clause 1. A method of determining hepatic clearance in a system comprising (i) a human patient comprising a native liver supported on (ii) an extracorporeal liver perfusion system (ECS) comprising an (iii) extracorporeal liver, the method comprising determining compartment volumes (L) of the patient systemic circulation(S), the extracorporeal liver perfusion system (E), the extracorporeal liver (X), and the human patient native liver (H);
   deriving or measuring blood flow rates q (L/min) between the compartments;
   obtaining blood or serum samples collected at one or more or two or more time points within 180 minutes of intravenous administration of a dose of a first distinguishable cholate compound (D_{IV}) to the systemic circulation of the patient(S), and optional simultaneous oral or nasogastric (NG) tube administration of a dose of a second distinguishable cholate compound (Dₒᵣₐₗ) to the patient;
   wherein the blood or serum samples were simultaneously collected from each of the patient systemic circulation (B_{S}); the extracorporeal liver perfusion system (B_{E}); and inferior vena cava (IVC) outflow of the EC liver (B_{X});
   measuring the first distinguishable cholate concentration and optionally the second distinguishable cholate concentration in each of the blood or serum samples; and
   calculating the simultaneous systemic clearance of the intravenously administered first distinguishable cholate of the patient liver (Cl_{H}) and of the extracorporeal liver (Clx) in the extracorporeal liver perfusion system (ECS).
Clause 2. The method of clause 1, further comprising
   calculating simultaneous portal clearance of the orally or nasogastric administered second distinguishable cholate of the patient liver (Cl_{H}) and of the extracorporeal liver (Clx) in the extracorporeal liver perfusion system (ECS).
Clause 3. The method of clause 1 or 2, wherein the blood or serum samples were collected at one or more or two or more time points within about 120 min, about 90 min, about 60 min, about 45 min, or about 20 minutes of administering the first distinguishable cholate compound to the systemic circulation of the patient(S) via intravenous administration.
Clause 4. The method of any one of clauses 1-3, wherein the deriving or measuring flow rates (L/min) between each of the compartments patient systemic circulation (S), patient human liver (H), ECS (E), and extracorporeal liver (X) comprises a system of first-order ordinary differential equations 1 to 4, respectively:

$$\frac{{dC}_{S}}{dt} = \frac{1}{{V}_{S}} ( - [ {q}_{SH} + {q}_{SE} ] {C}_{S} + {q}_{HS} \cdot {C}_{H} + {q}_{ES} \cdot {C}_{E} ) , \qquad Eqn . 1 ;$$

$$\frac{{dC}_{H}}{dt} = \frac{1}{{V}_{H}} ( - [ {Cl}_{H} + {q}_{HS} ] {C}_{H} + {q}_{SH} \cdot {C}_{S} ) , \qquad Eqn . 2 ;$$

$$\frac{{dC}_{E}}{dt} = \frac{1}{{V}_{E}} ( - [ {q}_{ES} + {q}_{EX} ] {C}_{E} + {q}_{XE} \cdot {C}_{X} + {q}_{SE} \cdot {C}_{S} ) , and \qquad Eqn . 3 ;$$

$$\frac{{dC}_{X}}{dt} = \frac{1}{{V}_{X}} ( - [ {Cl}_{X} + {q}_{XE} ] {C}_{X} + {q}_{EX} \cdot {C}_{E} ) , \qquad Eqn . 4 ,$$

wherein
V is the volume (L) in each of the patient systemic circulation (V_{S}), patient human liver (V_{H}), ECS (VE), and extracorporeal liver (V_{X}) compartments; C is the blood concentration (mM) of first distinguishable cholate in each of the patient systemic circulation (C_{S}), patient human liver (C_{H}), ECS (C_{E}), and extracorporeal liver (C_{X}) compartments; q is the blood flow rate between compartments (L/min); Cl_{H} is the hepatic clearance (L/min) of the human liver, and Cl_{X} is the hepatic clearance (L/min) of the extracorporeal liver.
Clause 5. The method of clause 4, wherein
   q_{SH} is patient systemic inflow to human liver (hepatic arterial inflow+portal venous inflow), q_{HS} is outflow from human liver back to systemic circulation, q_{SE} is inflow from patient systemic circulation to ECS, q_{ES} is outflow from ECS to patient systemic circulation, q_{XE} is inferior vena cava (IVC) blood outflow from EC liver back to ECS, q_{EX,PV} is portal venous (PV) blood inflow to EC liver from ECS, q_{EX,HA} is hepatic arterial inflow to EC liver from ECS, and q_{EX} is total blood inflow to EC liver from ECS.
Clause 6. A method of determining hepatic clearance in a system comprising (i) a human patient without a native liver supported on (ii) an extracorporeal liver perfusion system (ECS) comprising an (iii) extracorporeal liver, the method comprising
determining compartment volumes (L) of the patient systemic circulation(S), the extracorporeal liver perfusion system (E), and the extracorporeal liver (X);
deriving or measuring blood flow rates q (L/min) between the compartments;
obtaining blood or serum samples collected at one or more or two or more time points within 180 minutes of intravenous administration of a dose of a first distinguishable cholate compound (D_{IV}) to the systemic circulation of the patient(S), and optional simultaneous oral or nasogastric (NG) tube administration of a dose of a second distinguishable cholate compound (Dₒᵣₐₗ) to the patient;
wherein the blood or serum samples were simultaneously collected from each of the patient systemic circulation (B_{S}); the extracorporeal liver perfusion system (B_{E}); and inferior vena cava (IVC) outflow of the EC liver (B_{X});
measuring the first distinguishable cholate concentration and optionally the second distinguishable cholate concentration in each of the blood or serum samples; and
calculating the systemic clearance of the intravenously administered first distinguishable cholate of the extracorporeal liver (Clx) in the extracorporeal liver perfusion system (ECS).
Clause 7. The method of clause 6, further comprising
calculating simultaneous portal clearance of the orally or nasogastric administered second distinguishable cholate of the extracorporeal liver (Clx) in the extracorporeal liver perfusion system (ECS).
Clause 8. The method of clause 6 or 7, wherein the blood or serum samples were collected at one or more or two or more time points within about 120 min, about 90 min, about 60 min, about 45 min, or about 20 minutes of administering the first distinguishable cholate compound to the systemic circulation of the patient(S) via intravenous administration.
Clause 9. The method of any one of clauses 6-8, wherein the deriving or measuring flow rates (L/min) between each of the compartments patient systemic circulation(S), ECS (E), and extracorporeal liver (X) comprises a system of first-order ordinary differential equations 5 to 7, respectively:

$$\frac{{dC}_{S}}{dt} = \frac{1}{{V}_{S}} ( - {q}_{SE} {C}_{S} + {q}_{ES} \cdot {C}_{E} ) , \qquad Eqn . 5 ;$$

$$\frac{{dC}_{E}}{dt} = \frac{1}{{V}_{E}} ( - [ {q}_{ES} + {q}_{EX} ] {C}_{E} + {q}_{XE} \cdot {C}_{X} + {q}_{SE} \cdot {C}_{S} ) , and \qquad Eqn . 6 ;$$

$$\frac{{dC}_{X}}{dt} = \frac{1}{{V}_{X}} ( - [ {Cl}_{X} + {q}_{XE} ] {C}_{X} + {q}_{EX} \cdot {C}_{E} ) , \qquad Eqn . 7 ,$$

wherein
V is the volume (L) in each of the patient or BDRD systemic circulation (V_{S}), the ECS (V_{E}), and the extracorporeal liver (V_{X}) compartments; C is the blood concentration (mM) of first distinguishable cholate in each of the patient systemic circulation (C_{S}), ECS (C_{E}), and extracorporeal liver (C_{X}) compartments; q is the blood flow rate between compartments (L/min); and Cl_{X} is the hepatic clearance (L/min) of the extracorporeal liver.
Clause 10. The method of clause 9, wherein
q_{SE} is inflow from patient systemic circulation to ECS, q_{ES} is outflow from ECS to patient systemic circulation, q_{XE} is inferior vena cava (IVC) blood outflow from EC liver back to ECS, and q_{EX} is total blood inflow to EC liver from ECS.
Clause 11. A method of determining total systemic clearance (Cl_{Tot}) in a system comprising an extracorporeal liver perfusion system (ECS), a human patient having a native liver, and an extracorporeal (EC) liver, the method comprising:
receiving blood or serum samples collected from one or more of the ECS, the patient systemic circulation, and inferior vena cava (IVC) outflow of the EC liver at one or more, or two or more, time points within 180 minutes of intravenous administration to the patient of a first distinguishable cholate compound, and optional simultaneous oral or nasogastric administration to the patient of a second distinguishable cholate compound;
measuring distinguishable cholate concentrations (C) of the first distinguishable cholate compound and optionally the second distinguishable cholate compound in each of the blood or serum samples;
optionally measuring blood flow rates (q, L/min) in one or more of the ECS (E), the human patient systemic circulation(S), and the extracorporeal liver (X) in at least one of the time points; and
calculating total systemic clearance (Cl_{Tot}) as the intravenous clearance of first distinguishable cholate compound of the system.
Clause 12. The method of clause 11, wherein the calculating intravenous clearance of the first distinguishable cholate from the system comprises a method selected from the group consisting of SHUNT V1.0, SHUNT V1.1, and SHUNT V2.0 methods as disclosed herein.
Clause 13. The method of clause 11 or 12, further comprising calculating systemic clearance in the ECS (Cl_{E}) comprising
measuring the ECS blood flow rate (q, L/min) as blood inflow rate from patient systemic circulation to the ECS (q_{SE}, L/min); and
calculating the first distinguishable cholate compound clearance in the ECS (Cl_{E}) as the q_{SE} times extraction efficiency of the ECS (E_{E}) by Eqn 10:

$${Cl}_{E} = {q}_{SE} \cdot {E}_{E} , \qquad Eqn . 10.$$

Clause 14. The method of clause 13, wherein the extraction efficiency of the ECS (E_{E}) at one or more of the time points (t) is related to the concentration of the distinguishable cholate compound in the ECS and flow rates in and out of the ECS.
Clause 15. The method of clause 13 or 14, wherein the extraction efficiency of the ECS (E_{E}) is calculated at the one or more of the time points (t) comprising Eqn. 8:

$${E}_{E} ( t ) = \frac{{C}_{S} ( t ) \cdot {q}_{SE} ( t ) - {C}_{E} ( t ) \cdot {q}_{ES} ( t )}{{C}_{S} ( t ) \cdot {q}_{SE} ( t )} , \qquad Eqn . 8$$

wherein
C_{E}(t) is the concentration of the first distinguishable cholate compound at time (t) in the ECS;
q_{ES}(t) is the outflow rate at time (t) from the ECS to the patient systemic circulation;
C_{S}(t) is the concentration of first distinguishable cholate compound in the patient systemic circulation at time (t); and
q_{SE}(t) is the inflow rate from the patient systemic circulation to the ECS at time (t).
Clause 16. The method of any one of clauses 11-15, further comprising calculating systemic clearance of the extracorporeal (EC) liver comprising
measuring the EC liver blood flow rate (q, L/min) as the IVC blood outflow rate from the extracorporeal (EC) liver back to ECS (q_{XE}); and
calculating the systemic clearance of the extracorporeal liver (Cl_{X}) as q_{XE} times the extraction efficiency of the extracorporeal liver (E_{X}) by Eqn. 11:

$${Cl}_{X} = {q}_{XE} \cdot {E}_{X} . \qquad Eqn . 11$$

Clause 17. The method of clause 16, wherein the extraction efficiency of the EC liver (E_{X}) is calculated at one or more of the time points (t) comprising Eqn. 9:

$${E}_{X} ( t ) = \frac{{C}_{E} ( t ) \cdot {q}_{EX} ( t ) - {C}_{X} ( t ) \cdot {q}_{XE} ( t )}{{C}_{E} ( t ) \cdot {q}_{EX} ( t )} , \qquad Eqn . 9$$

wherein
C_{E}(t) is the concentration of the first distinguishable cholate compound at time (t) in the ECS;
q_{EX}(t) is the total blood inflow to EC liver from the ECS at time (t);
C_{X}(t) is the concentration of the first distinguishable cholate compound at time (t) in the IVC outflow of the EC liver; and
q_{XE}(t) is IVC blood outflow from EC liver back to ECS.
Clause 18. The method of any one of clauses 11-17, further comprising
calculating the systemic clearance of the human liver (Cl_{H}) as the total clearance (Cl_{Tot}) minus the clearance of the ECS (Cl_{E}) comprising Eqn. 12:

$${Cl}_{H} = {Cl}_{Tot} - {Cl}_{E} . \qquad Eqn . 12$$

Clause 19. The method of any one of clauses 11-18, further comprising in one of more of the ECS system, the patient, and the extracorporeal liver
estimating an area under the curve of the blood or serum concentrations of the intravenously administered distinguishable cholate compound (AUCiv); and
optionally estimating an area under the curve of the blood or serum concentrations of the orally or NG administered distinguishable cholate compound (AUCoral).
Clause 20. The method of clause 19, wherein the estimating the AUCiv in one of more of the ECS system, the patient, and the extracorporeal liver comprises exponential fitting the intravenous concentration data to a systemic cholate clearance curve comprising fast, moderate, and slow phases of clearance over about 180 min or more after the administration of the intravenous dose.
Clause 21. The method of clause 19 or 20, further comprising calculating systemic hepatic filtration rate (HFRs) in one of more of the ECS system, the patient, and the extracorporeal liver from the measured systemic clearance adjusted for body weight (units mL min⁻¹ kg⁻¹) by Equation 26,

$${HFR}_{S} = \frac{{D}_{IV}}{{AUC}_{IV} \cdot BW} , \qquad Eqn . 26$$

where D_{IV} is the administered intravenous dose of the first distinguishable cholate, and BW is the patient body weight.
Clause 22. The method of any one of clauses 19-21, further comprising calculating portal hepatic filtration rate (HFRp) in one of more of the ECS system, the patient, and the extracorporeal liver from the measured systemic clearance body weight (units mL min⁻¹ kg⁻¹) by Equation 25:

$${HFR}_{P} = \frac{{D}_{PO}}{{AUC}_{Oral} \cdot BW} , \qquad Eqn . 25$$

where D_{PO} is the administered oral or nasogastric cholate dose of second distinguishable cholate, and BW is the patient body weight.
Clause 23. The method of any one of clauses 1-22, wherein the first and optional second distinguishable cholate compounds are independently distinguishable cholic acids, cholic acid conjugates, or cholic acid analogs, wherein the first and optional second distinguishable cholate compounds are different.
Clause 24. The method of any one of clauses 1-23, wherein the first and optional second distinguishable cholic acids are independently isotopically labeled cholic acids.
Clause 25. The method of clause 24, wherein the isotopically labeled cholic acids are stable isotope labeled cholic acids.
Clause 26. The method of clause 25, wherein the stable isotope labeled cholic acids are selected from the group consisting of cholic acid-2,2,4,4-D4 (D4-CA; CA-D4), 24-¹³C-cholic acid (13C-CA), 2,2,3,4,4-d₅ cholic acid (D₅-CA), and 3,6,6,7,8,11,11,12-d8 cholic acid (D8-CA).

The above specification, examples and data provide a complete description of the manufacture and use of the composition of the invention. Since many embodiments of the invention can be made without departing from the spirit and scope of the invention, the invention resides in the claims hereinafter appended.

### REFERENCES

ADDIN EN.REFLIST 1. McRae M P, Helmke S M, Burton J R, Jr., Everson G T. Compartmental model describing the physiological basis for the HepQuant SHUNT test. Transl Res. 2023; 252:53-63. doi: 10.1016/j.trsl.2022.08.002
2. McRae M P, Kittelson J, Helmke S M, Everson G T. Advances in noninvasive measurement of liver function and physiology: The HepQuant DuO test. Basic Clin Pharmacol Toxicol. 2024; 134(3):385-395. doi: 10.1111/bcpt.13980
3. McRae M P, Kittelson J, Helmke S M, Everson G T. Within Individual Reproducibility of a Dual Sample Oral Cholate Challenge Test (DuO) and Other Simplified Versions of the HepQuant Test. Clin Transl Sci. 2024; 17(4):e13786. doi:10.1111/cts.13786
4. Lemmens H J, Bernstein D P, Brodsky J B. Estimating blood volume in obese and morbidly obese patients. Obes Surg. 2006; 16(6):773-6. doi: 10.1381/096089206777346673
5. Andersen V, Sonne J, Sletting S, Prip A. The volume of the liver in patients correlates to body weight and alcohol consumption. Alcohol Alcohol. 2000; 35(5):531-532. doi: 10.1093/alcalc/35.5.531
6. Yu H C, You H, Lee H, Jin Z W, Moon J I, Cho B H. Estimation of standard liver volume for liver transplantation in the Korean population. Liver Transpl. June 2004; 10(6):779-83. doi: 10.1002/lt.20188
7. Heinemann A, Wischhusen F, Püschel K, Rogiers X. Standard liver volume in the Caucasian population. Liver Transpl. September 1999; 5(5):366-8. doi: 10.1002/lt.500050516
8. Eipel C, Abshagen K, Vollmar B. Regulation of hepatic blood flow: the hepatic arterial buffer response revisited. World J Gastroenterol. 2010; 16(48):6046-6057. doi: 10.3748/wjg.v16.i48.6046
9. Lautt W W. Hepatic vasculature: a conceptual review. Gastroenterology. 1977/11/01/1977; 73(5): 1163-1169. doi: doi.org/10.1016/S0016-5085(19)31879-7
10. Rappaport A M. Hepatic blood flow: morphologic aspects and physiologic regulation. Int Rev Physiol. 1980; 21:1-63.

1. A method of determining hepatic clearance in a system comprising (i) a human patient comprising a native liver supported on (ii) an extracorporeal liver perfusion system (ECS) comprising an (iii) extracorporeal liver, the method comprising
determining compartment volumes (L) of the patient systemic circulation(S), the extracorporeal liver perfusion system (E), the extracorporeal liver (X), and the human patient native liver (H);
deriving or measuring blood flow rates q (L/min) between the compartments;
obtaining blood or serum samples collected at one or more or two or more time points within 180 minutes of intravenous administration of a dose of a first distinguishable cholate compound (D_{IV}) to the systemic circulation of the patient (S), and optional simultaneous oral or nasogastric (NG) tube administration of a dose of a second distinguishable cholate compound (Dₒᵣₐₗ) to the patient;
wherein the blood or serum samples were simultaneously collected from each of the patient systemic circulation (B_{S}); the extracorporeal liver perfusion system (B_{E}); and inferior vena cava (IVC) outflow of the EC liver (B_{X});
measuring the first distinguishable cholate concentration and optionally the second distinguishable cholate concentration in each of the blood or serum samples; and
calculating the simultaneous systemic clearance of the intravenously administered first distinguishable cholate of the patient liver (Cl_{H}) and of the extracorporeal liver (Clx) in the extracorporeal liver perfusion system (ECS).

2. The method of claim 1, further comprising
calculating simultaneous portal clearance of the orally or nasogastric administered second distinguishable cholate of the patient liver (Cl_{H}) and of the extracorporeal liver (Clx) in the extracorporeal liver perfusion system (ECS).

3. The method of claim 1, wherein the blood or serum samples were collected at one or more or two or more time points within about 120 min, about 90 min, about 60 min, about 45 min, or about 20 minutes of administering the first distinguishable cholate compound to the systemic circulation of the patient (S) via intravenous administration.

4. The method of claim 1, wherein the deriving or measuring flow rates (L/min) between each of the compartments patient systemic circulation (S), patient human liver (H), ECS (E), and extracorporeal liver (X) comprises a system of first-order ordinary differential equations 1 to 4, respectively:
$$\frac{{dC}_{S}}{dt} = \frac{1}{{V}_{S}} ( - ⌊ {q}_{SH} + {q}_{SE} ⌋ {C}_{S} + {q}_{HS} \cdot {C}_{H} + {q}_{ES} \cdot {C}_{E} ) ; \qquad Eqn . 1$$
$$\frac{{dC}_{H}}{dt} = \frac{1}{{V}_{H}} ( - ⌈ {Cl}_{H} + {q}_{HS} ⌉ {C}_{H} + {q}_{SH} \cdot {C}_{S} ) ; \qquad Eqn . 2$$
$$\frac{{dC}_{E}}{dt} = \frac{1}{{V}_{E}} ( - [ {q}_{ES} + {q}_{EX} ] {C}_{E} + {q}_{XE} \cdot {C}_{X} + {q}_{SE} \cdot {C}_{S} ) ; and \qquad Eqn . 3$$
$$\frac{{dC}_{X}}{dt} = \frac{1}{{V}_{X}} ( - [ {Cl}_{X} + {q}_{XE} ] {C}_{X} + {q}_{EX} \cdot {C}_{E} ) , \qquad Eqn . 4$$
wherein
V is the volume (L) in each of the patient systemic circulation (V_{S}), patient human liver (V_{H}), ECS (VE), and extracorporeal liver (Vx) compartments; C is the blood concentration (μM) of first distinguishable cholate in each of the patient systemic circulation (C_{S}), patient human liver (C_{H}), ECS (C_{E}), and extracorporeal liver (C_{X}) compartments; q is the blood flow rate between compartments (L/min); Cl_{H} is the hepatic clearance (L/min) of the human liver, and Cl_{X} is the hepatic clearance (L/min) of the extracorporeal liver.

5. The method of claim 4, wherein
q_{SH} is patient systemic inflow to human liver (hepatic arterial inflow+portal venous inflow), q_{HS} is outflow from human liver back to systemic circulation, q_{SE} is inflow from patient systemic circulation to ECS, q_{ES} is outflow from ECS to patient systemic circulation, q_{XE} is inferior vena cava (IVC) blood outflow from EC liver back to ECS, q_{EX,PV} is portal venous (PV) blood inflow to EC liver from ECS, q_{EX,HA} is hepatic arterial inflow to EC liver from ECS, and q_{EX} is total blood inflow to EC liver from ECS.

6. A method of determining hepatic clearance in a system comprising (i) a human patient without a native liver supported on (ii) an extracorporeal liver perfusion system (ECS) comprising an (iii) extracorporeal liver, the method comprising
determining compartment volumes (L) of the patient systemic circulation(S), the extracorporeal liver perfusion system (E), and the extracorporeal liver (X);
deriving or measuring blood flow rates q (L/min) between the compartments;
obtaining blood or serum samples collected at one or more or two or more time points within 180 minutes of intravenous administration of a dose of a first distinguishable cholate compound (D_{IV}) to the systemic circulation of the patient (S), and optional simultaneous oral or nasogastric (NG) tube administration of a dose of a second distinguishable cholate compound (Doral) to the patient;
wherein the blood or serum samples were simultaneously collected from each of the patient systemic circulation (B_{S}); the extracorporeal liver perfusion system (B_{E}); and inferior vena cava (IVC) outflow of the EC liver (B_{X});
measuring the first distinguishable cholate concentration and optionally the second distinguishable cholate concentration in each of the blood or serum samples; and
calculating the systemic clearance of the intravenously administered first distinguishable cholate of the extracorporeal liver (Clx) in the extracorporeal liver perfusion system (ECS).

7. The method of claim 6, further comprising
calculating simultaneous portal clearance of the orally or nasogastric administered second distinguishable cholate of the extracorporeal liver (Clx) in the extracorporeal liver perfusion system (ECS).

8. The method of claim 6, wherein the blood or serum samples were collected at one or more or two or more time points within about 120 min, about 90 min, about 60 min, about 45 min, or about 20 minutes of administering the first distinguishable cholate compound to the systemic circulation of the patient(S) via intravenous administration.

9. The method of claim 6, wherein the deriving or measuring flow rates (L/min) between each of the compartments patient systemic circulation (S), ECS (E), and extracorporeal liver (X) comprises a system of first-order ordinary differential equations 5 to 7, respectively:
$$\frac{{dC}_{S}}{dt} = \frac{1}{{V}_{S}} ( - {q}_{SE} {C}_{S} + {q}_{ES} \cdot {C}_{E} ) ; \qquad Eqn . 5$$
$$\frac{{dC}_{E}}{dt} = \frac{1}{{V}_{E}} ( - [ {q}_{ES} + {q}_{EX} ] {C}_{E} + {q}_{XE} \cdot {C}_{X} + {q}_{SE} \cdot {C}_{S} ) ; and \qquad Eqn . 6$$
$$\frac{{dC}_{X}}{dt} = \frac{1}{{V}_{X}} ( - [ {Cl}_{X} + {q}_{XE} ] {C}_{X} + {q}_{EX} \cdot {C}_{E} ) , \qquad Eqn . 7$$
wherein
V is the volume (L) in each of the patient or BDRD systemic circulation (V_{S}), the ECS (V_{E}), and the extracorporeal liver (V_{X}) compartments; C is the blood concentration (μM) of first distinguishable cholate in each of the patient systemic circulation (C_{S}), ECS (C_{E}), and extracorporeal liver (C_{X}) compartments; q is the blood flow rate between compartments (L/min); and Cl_{X} is the hepatic clearance (L/min) of the extracorporeal liver.

10. The method of claim 9, wherein
q_{SE} is inflow from patient systemic circulation to ECS, q_{ES} is outflow from ECS to patient systemic circulation, q_{XE} is inferior vena cava (IVC) blood outflow from EC liver back to ECS, and q_{EX} is total blood inflow to EC liver from ECS.

11. A method of determining total systemic clearance (Cl_{Tot}) in a system comprising an extracorporeal liver perfusion system (ECS), a human patient having a native liver, and an extracorporeal (EC) liver, the method comprising:
receiving blood or serum samples collected from one or more of the ECS, the patient systemic circulation, and inferior vena cava (IVC) outflow of the EC liver at one or more, or two or more, time points within 180 minutes of intravenous administration to the patient of a first distinguishable cholate compound, and optional simultaneous oral or nasogastric administration to the patient of a second distinguishable cholate compound;
measuring distinguishable cholate concentrations (C) of the first distinguishable cholate compound and optionally the second distinguishable cholate compound in each of the blood or serum samples;
optionally measuring blood flow rates (q, L/min) in one or more of the ECS (E), the human patient systemic circulation(S), and the extracorporeal liver (X) in at least one of the time points; and
calculating total systemic clearance (Cl_{Tot}) as the intravenous clearance of first distinguishable cholate compound of the system.

12. The method of claim 11, wherein the calculating intravenous clearance of the first distinguishable cholate from the system comprises a method selected from the group consisting of SHUNT V1.0, SHUNT V1.1, and SHUNT V2.0 methods as disclosed herein.

13. The method of claim 11, further comprising calculating systemic clearance in the ECS (Cl_{E}) comprising
measuring the ECS blood flow rate (q, L/min) as blood inflow rate from patient systemic circulation to the ECS (q_{SE}, L/min); and
calculating the first distinguishable cholate compound clearance in the ECS (Cl_{E}) as the q_{SE} times extraction efficiency of the ECS (E_{E}) by Eqn 10:
$${Cl}_{E} = {q}_{SE} \cdot {E}_{E} . \qquad Eqn . 10$$

14. The method of claim 13, wherein the extraction efficiency of the ECS (E_{E}) at one or more of the time points (t) is related to the concentration of the distinguishable cholate compound in the ECS and flow rates in and out of the ECS.

15. The method of claim 13, wherein the extraction efficiency of the ECS (E_{E}) is calculated at the one or more of the time points (t) comprising Eqn. 8:
$${E}_{E} ( t ) = \frac{{C}_{S} ( t ) \cdot {q}_{SE} ( t ) - {C}_{E} ( t ) \cdot {q}_{ES} ( t )}{{C}_{S} ( t ) \cdot {q}_{SE} ( t )} , \qquad Eqn . 8$$
wherein
C_{E}(t) is the concentration of the first distinguishable cholate compound at time (t) in the ECS;
q_{ES}(t) is the outflow rate at time (t) from the ECS to the patient systemic circulation;
C_{S}(t) is the concentration of first distinguishable cholate compound in the patient systemic circulation at time (t); and
q_{SE} (t) is the inflow rate from the patient systemic circulation to the ECS at time (t).

16. The method of claim 11, further comprising calculating systemic clearance of the extracorporeal (EC) liver comprising
measuring the EC liver blood flow rate (q, L/min) as the IVC blood outflow rate from the extracorporeal (EC) liver back to ECS (q_{XE}); and
calculating the systemic clearance of the extracorporeal liver (Cl_{X}) as q_{XE} times the extraction efficiency of the extracorporeal liver (E_{X}) by Eqn. 11:
$${Cl}_{X} = {q}_{XE} \cdot {E}_{X} . \qquad Eqn . 11$$

17. The method of claim 16, wherein the extraction efficiency of the EC liver (E_{X}) is calculated at one or more of the time points (t) comprising Eqn. 9:
$${E}_{X} ( t ) = \frac{{C}_{E} ( t ) \cdot {q}_{EX} ( t ) - {C}_{X} ( t ) \cdot {q}_{XE} ( t )}{{C}_{E} ( t ) \cdot {q}_{EX} ( t )} , \qquad Eqn . 9$$
wherein
C_{E}(t) is the concentration of the first distinguishable cholate compound at time (t) in the ECS;
q_{EX}(t) is the total blood inflow to EC liver from the ECS at time (t);
C_{X}(t) is the concentration of the first distinguishable cholate compound at time (t) in the IVC outflow of the EC liver; and
q_{XE}(t) is IVC blood outflow from EC liver back to ECS.

18. The method of claim 11, further comprising
calculating the systemic clearance of the human liver (Cl_{H}) as the total clearance (Cl_{Tot}) minus the clearance of the ECS (Cl_{E}) comprising Eqn. 12:
$${Cl}_{H} = {Cl}_{Tot} - {Cl}_{E} . \qquad Eqn . 12$$

19. The method of claim 11, further comprising in one of more of the ECS system, the patient, and the extracorporeal liver
estimating an area under the curve of the blood or serum concentrations of the intravenously administered distinguishable cholate compound (AUCiv); and
optionally estimating an area under the curve of the blood or serum concentrations of the orally or NG administered distinguishable cholate compound (AUCoral).

20. The method of claim 19, wherein the estimating the AUCiv in one of more of the ECS system, the patient, and the extracorporeal liver comprises exponential fitting the intravenous concentration data to a systemic cholate clearance curve comprising fast, moderate, and slow phases of clearance over about 180 min or more after the administration of the intravenous dose.

21. The method of claim 19, further comprising calculating systemic hepatic filtration rate (HFRs) in one of more of the ECS system, the patient, and the extracorporeal liver from the measured systemic clearance adjusted for body weight (units mL min⁻¹ kg⁻¹) by Equation 26,
$${HFR}_{S} = \frac{{D}_{IV}}{{AUC}_{IV} \cdot BW} , \qquad Eqn . 26$$
where D_{IV} is the administered intravenous dose of the first distinguishable cholate, and BW is the patient body weight.

22. The method of claim 19, further comprising calculating portal hepatic filtration rate (HFRₚ) in one of more of the ECS system, the patient, and the extracorporeal liver from the measured systemic clearance body weight (units mL min⁻¹ kg⁻¹) by Equation 25:
$${HFR}_{P} = \frac{{D}_{PO}}{{AUC}_{Oral} \cdot BW} , \qquad Eqn . 25$$
where D_{PO} is the administered oral or nasogastric cholate dose of second distinguishable cholate, and BW is the patient body weight.

23. The method of claim 1, wherein the first and optional second distinguishable cholate compounds are independently distinguishable cholic acids, cholic acid conjugates, or cholic acid analogs, wherein the first and optional second distinguishable cholate compounds are different.

24. The method of claim 23, wherein the first and optional second distinguishable cholic acids are independently isotopically labeled cholic acids.

25. The method of claim 24, wherein the isotopically labeled cholic acids are stable isotope labeled cholic acids.

26. The method of claim 25, wherein the stable isotope labeled cholic acids are selected from the group consisting of cholic acid-2,2,4,4-D4 (D4-CA; CA-D4), 24-¹³C-cholic acid (¹³C-CA), 2,2,3,4,4-d₅ cholic acid (D₅-CA), and 3,6,6,7,8,11,11,12-d8 cholic acid (D8-CA).
